(19) United States
(12) Patent Application Publication
(10) Pub. No.: US 2022/0112081 A1
(43) Pub. Date: Apr. 14, 2022

(54) PROCESS FOR PRODUCING HYDROGEN-LEAN SYNGAS FOR SYNTHESIS PROCESSES

(71) Applicant: ENI S.p.A., Roma (IT)

(72) Inventors: Vijayanand RAJAGOPALAN, Bengaluru (IN); Atul PANT, Bengaluru (IN); Ravichander NARAYANASWAMY, Bengaluru (IN)

(21) Appl. No.: 17/426,499

(22) PCT Filed: Jan. 2, 2020
(86) PCT No.: PCT/IB2020/050014

§ 371 Date: Jul. 28, 2021

Related U.S. Application Data
(60) Provisional application No. 62/797,587, filed on Jan. 28, 2019.

(51) Int. Cl. C01B 3/38; B01J 19/24; C07C 51/10; C07C 41/01; C07C 45/50; C07C 27/20

(52) U.S. Cl. CPC C01B 3/382; C01B 2203/1247; B01J 19/245; C07C 51/10; C07C 41/01; C07C 45/50; C07C 27/20; C01B 2203/0261; C01B 2203/168; C01B 2203/061; C01B 2203/062; C01B 2203/1614; C01B 2203/1628; C01B 2203/1241; C01B 3/386

(57) ABSTRACT

A process for producing hydrogen-lean syngas includes the steps of reacting, via a catalytic partial oxidation (CPO) reaction, a CPO reactant mixture in a CPO reactor to produce the hydrogen-lean syngas, wherein the CPO reactant mixture includes hydrocarbons and oxygen. The hydrocarbons include greater than or equal to about 3 mol % C2+ alkanes, wherein the CPO reactor include a CPO catalyst, and wherein the hydrogen-lean syngas include hydrogen, carbon monoxide, carbon dioxide, water, and unreacted hydrocarbons The hydrogen-lean syngas is characterized by a molar ratio of hydrogen to carbon monoxide (H2/CO) in a range of from about 0.8 to about 1.6. A system for carrying out the process is also provided.

### TECHNICAL FIELD

The present disclosure relates to methods of producing hydrogen-lean synthesis gas (e.g., having a molar ratio of hydrogen to carbon monoxide (H₂/CO) in a range of from about 0.8 to 1.6); more specifically, the present disclosure methods of producing hydrogen-lean synthesis gas via catalytic partial oxidation (CPO); still more specifically, the present disclosure relates to methods of producing hydrogen-lean synthesis gas via catalytic partial oxidation (CPO) of a CPO reactant mixture comprising hydrocarbons and oxygen, wherein the hydrocarbons comprise greater than or equal to about 3 mole percent (mol %) of higher hydrocarbons (e.g., alkanes comprising 2 or more carbons, C₂₊).

### BACKGROUND

Synthesis gas (syngas) is a mixture comprising carbon monoxide (CO) and hydrogen (H₂), as well as small amounts of carbon dioxide (CO₂), water (H₂O), and unreacted methane (CH₄). Syngas is generally used as an intermediate in a variety of synthesis processes, including, without limitation, dimethyl ether (DME), alcohols, such as methanol, ethanol, oxoalcohols (e.g., n-butanol etc.), ethylene glycol, aldehydes, and the like. Syngas is produced conventionally by steam reforming of natural gas (steam methane reforming or SMR), although other hydrocarbon sources can be used for syngas production, such as refinery off-gases, naphtha feedstocks, heavy hydrocarbons, coal, biomass, etc. SMR is an endothermic process and requires significant energy input to drive the reaction forward. Conventional endothermic technologies such as SMR produce syngas with a hydrogen content greater than the required for a variety of downstream chemical syntheses.

In an autothermal reforming (ATR) process, a portion of the natural gas is burned as fuel to drive the conversion of natural gas to syngas resulting in relatively low hydrogen and high CO₂ concentrations. Conventional combined reforming (CR) technology pairs SMR with autothermal reforming (ATR) to reduce the amount of hydrogen present in syngas. ATR produces a syngas with a lower hydrogen content. CR syngas generally has a hydrogen content greater than needed for many downstream synthesis processes. Furthermore, SMR is a highly endothermic process, and the endothermicity of the SMR technology requires burning fuel to drive the syngas synthesis. Consequently, the SMR technology reduces the energy efficiency of the downstream chemical synthesis process.

Syngas can also be produced (non-commercially) by catalytic partial oxidation (CPO or CPOx) of natural gas. CPO processes employ partial oxidation of hydrocarbon feeds to syngas comprising CO and H₂. The CPO process is exothermic, thus eliminating the need for external heat supply. Conventional partial oxidation processes do not produce hydrogen-lean synthesis suitable for use in downstream syntheses requiring molar ratios of hydrogen to carbon monoxide less than about 1.6. Thus, there is an ongoing need for the development of methods for syngas production via CPO processes that provide a hydrogen-lean synthesis gas for a variety of downstream syntheses.

### BRIEF DESCRIPTION OF THE DRAWINGS

For a detailed description of the preferred embodiments of the disclosed methods, reference will now be made to the accompanying drawing in which:

FIG. 1 is a schematic of a chemical production system I for the production of hydrogen-lean synthesis gas via catalytic partial oxidation, according to embodiments of this disclosure;

FIG. 2 is a plot of the molar ratio of carbon monoxide to hydrogen (CO/H₂) in syngas from CPO as a function of reactor temperature without CO₂ injection in the reactant feed;

FIG. 3 is a plot of the molar ratio of carbon monoxide to hydrogen (CO/H₂) in syngas from CPO as a function of reactor temperature with CO₂ injection for a reactant feed comprising a molar ratio of carbon dioxide to methane (CO₂/CH₄) of 0.5;

FIG. 4 is a plot of the molar ratio of carbon monoxide to hydrogen (CO/H₂) in syngas from CPO as a function of reactor temperature with CO₂ injection for a reactant feed comprising a molar ratio of carbon dioxide to methane (CO₂/CH₄) of 1;

FIG. 5 is a plot showing the molar ratio of carbon monoxide to hydrogen (H₂/CO) in syngas from CPO as a function of the conversion (%) and the molar ratio of carbon dioxide to carbon (CO₂/C) in the reactant feed (in legend) at a pressure of 30 bar and an oxygen to carbon molar ratio (O₂/C) of 0.55;

FIG. 6 is a plot showing the molar ratio of carbon monoxide to hydrogen (H₂/CO) in syngas from CPO as a function of the conversion (%) and the molar ratio of carbon dioxide to carbon (CO₂/C) in the reactant feed (in legend) at a pressure of 75 bar and an oxygen to carbon molar ratio (O₂/C) of 0.55;

FIG. 7 is a plot showing the molar ratio of carbon monoxide to hydrogen (H₂/CO) in syngas from CPO as a function of the conversion (%) and the molar ratio of hydrocarbons having three carbons (C₃) to carbon (C₃/C) in the reactant feed (in legend) at a pressure of 75 bar, an oxygen to carbon molar ratio (O₂/C) of 0.55, and a carbon dioxide to carbon (CO₂/C) molar ratio of 0.25;

FIG. 8 is a plot showing the molar ratio of carbon monoxide to hydrogen (H₂/CO) in syngas from CPO as a function of the conversion (%) and the molar ratio of hydrocarbons having three carbons (C₃) to carbon (C₃/C) in the reactant feed (in legend) at a pressure of 75 bar, an oxygen to carbon molar ratio (O₂/C) of 0.55, and without CO₂ in the reactant feed;

FIG. 9 is a plot showing the molar ratio of carbon monoxide to hydrogen (H₂/CO) in syngas from CPO as a function of the conversion (%) and the molar ratio of hydrocarbons having two carbons (C₂) to carbon (C₂/C) in the reactant feed (in legend) at a pressure of 75 bar, an oxygen to carbon molar ratio (O₂/C) of 0.55, and a carbon dioxide to carbon (CO₂/C) molar ratio of 0.25;

FIG. 10 is a plot showing the molar ratio of carbon monoxide to hydrogen (H₂/CO) in syngas from CPO as a function of the conversion (%) and the molar ratio of hydrocarbons having two carbons (C₂) to carbon (C₂/C) in the reactant feed (in legend) at a pressure of 75 bar, an oxygen to carbon molar ratio (O₂/C) of 0.55, and without CO₂ in the reactant feed;

FIG. 11 is a plot showing the molar ratio of carbon monoxide to hydrogen (H₂/CO) in syngas from CPO as a function of the conversion (%) and the molar ratio of hydrocarbons having four carbons (C₄) to carbon (C₄/C) in the reactant feed (in legend) at a pressure of 75 bar, an oxygen to carbon molar ratio (O₂/C) of 0.55, and a carbon dioxide to carbon (CO₂/C) molar ratio of 0.25; and

FIG. 12 is a plot showing the molar ratio of carbon monoxide to hydrogen (H₂/CO) in syngas from CPO as a function of the conversion (%) and the molar ratio of hydrocarbons having four carbons (C₄) to carbon (C₄/C) in the reactant feed (in legend) at a pressure of 75 bar, an oxygen to carbon molar ratio (O₂/C) of 0.55, and without CO₂ in the reactant feed.

### DETAILED DESCRIPTION

The synthesis gas feeds for a variety of chemical synthesis processes require hydrogen-lean synthesis gas having a molar ratio of hydrogen to carbon monoxide (H₂/CO) of about 1:1. When the synthesis gas is produced from conventional reforming processes that provide synthesis gas having a higher molar ratio (e.g., about 2:1), the synthesis gas has to be pretreated, for example via a hydrogen removal unit (e.g., a pressure swing adsorption PSA unit), to reduce the molar ratio of H₂/CO of the synthesis gas. Conventional partial oxidation (POx) processes do not provide syngas having a H₂/CO molar ratio of about 1:1. The use of an intermediate hydrogen removal (e.g., PSA) step increases energy and capital cost requirements.

According to this disclosure, hydrogen-lean syngas (e.g., syngas having a molar ratio of H₂/CO in the range of from about 0.8 to about 1.6) can be produced via a catalytic partial oxidation (CPO) process. Via embodiments of the herein disclosed system and method, a CPO process can be tailored to provide a hydrogen-lean syngas having a desired composition (e.g., a reduced H₂/CO molar ratio relative to that of a syngas produced by a conventional POx process). Accordingly, the herein disclosed systems and methods can reduce the size of or eliminate hydrogen removal apparatus, thus reducing the number of unit operations, and thus can, in embodiments, also reduce energy requirements for the process.

In embodiments, CPO is utilized to produce a hydrogen-lean synthesis gas by utilizing a CPO reactant feed mixture that comprises higher hydrocarbons and/or carbon dioxide (CO₂). The use of reactant feed mixtures comprising higher hydrocarbons can allow for a reduction of the amount of CO₂ required to reach an H₂/CO molar ratio of about 1, and at the same time enable for production of hydrogen-lean syngas having the desired H₂/CO molar ratio of about 1 at a higher conversion of hydrocarbon to syngas.

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an embodiment,” “another embodiment,” “other embodiments,” “some embodiments,” and so forth, means that a particular element (e.g., feature, structure, property, and/or characteristic) described in connection with the embodiment is included in at least an embodiment described herein, and may or may not be present in other embodiments. In addition, it is to be understood that the described element(s) can be combined in any suitable manner in the various embodiments.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result. As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art. Compounds are described herein using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through the carbon of the carbonyl group. As used herein, the terms “Cₓ hydrocarbons” and “Cₓs” are interchangeable and refer to any hydrocarbon having x number of carbon atoms (C). For example, the terms “C₄ hydrocarbons” and “C₄s” both refer to any hydrocarbons having exactly 4 carbon atoms, such as n-butane, iso-butane, cyclobutane, 1-butene, 2-butene, isobutylene, butadiene, and the like, or combinations thereof.

As used herein, the term “Cₓ₊ hydrocarbons” refers to any hydrocarbon having greater than or equal to x carbon atoms (C). For example, the term “C₂₊ hydrocarbons” refers to any hydrocarbons having 2 or more carbon atoms, such as ethane, ethylene, C₃s, C₄s, C₅s, etc.

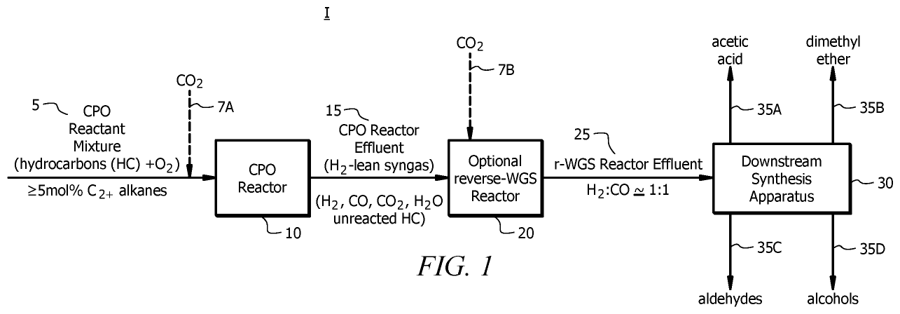

Referring to FIG. 1, a chemical production system I is disclosed. The chemical production system I generally comprises a catalytic partial oxidation (CPO or CPOx) reactor 10 and a downstream synthesis apparatus 30. Chemical production system I can further comprise a reverse water gas shift (r-WGS) reactor 20, in embodiments. As will be appreciated by one of skill in the art, and with the help of this disclosure, chemical production system components shown in FIG. 1 can be in fluid communication with each other (as represented by the connecting lines indicating a direction of fluid flow) through any suitable conduits (e.g., pipes, streams, etc.).

In embodiments, a process as disclosed herein can comprise a step of reacting, via a catalytic partial oxidation (CPO) reaction, a CPO reactant mixture 5 in a CPO reactor 10 to produce a hydrogen-lean syngas; wherein the CPO reactant mixture comprises hydrocarbons and oxygen and optionally carbon dioxide (CO₂); wherein the hydrocarbons comprise greater than or equal to about 3 mol % C₂₊ alkanes; wherein the CPO reactor comprises a CPO catalyst; wherein the hydrogen-lean syngas comprises hydrogen, carbon monoxide, carbon dioxide, water, and unreacted hydrocarbons; and wherein the hydrogen-lean syngas is characterized by a hydrogen to carbon monoxide (H₂/CO) molar ratio of from about 0.8 to about 1.6.

Generally, the CPO reaction is based on partial combustion of fuels, such as various hydrocarbons, and in the case of methane, CPO can be represented by equation (1):

CH₄+½O₂→CO+2H₂ (1)

Without wishing to be limited by theory, side reactions can take place along with the CPO reaction depicted in equation (1); and such side reactions can produce carbon dioxide (CO₂) and water (H₂O), for example via hydrocarbon combustion, which is an exothermic reaction. As will be appreciated by one of skill in the art, and with the help of this disclosure, and without wishing to be limited by theory, the CPO reaction as represented by equation (1) can yield a syngas with a hydrogen to carbon monoxide (H₂/CO) molar ratio having the theoretical stoichiometric limit of 2.0. Without wishing to be limited by theory, the theoretical stoichiometric limit of 2.0 for the H₂/CO molar ratio means that the CPO reaction as represented by equation (1) yields 2 moles of H₂ for every 1 mole of CO, i.e., H₂/CO molar ratio of (2 moles H₂/1 mole CO)=2. As will be appreciated by one of skill in the art, and with the help of this disclosure, the theoretical stoichiometric limit of 2.0 for the H₂/CO molar ratio in a CPO reaction cannot be achieved practically because reactants (e.g., hydrocarbons, oxygen) as well as products (e.g., H₂, CO) undergo side reactions at the conditions used for the CPO reaction. As will be appreciated by one of skill in the art, and with the help of this disclosure, and without wishing to be limited by theory, in the presence of oxygen, CO and H₂ can be oxidized to CO₂ and H₂O, respectively. The relative amounts (e.g., composition) of CO, H₂, CO₂ and H₂O can be further altered by the equilibrium of the water-gas shift (WGS) reaction, which will be discussed in more detail later herein. The side reactions that can take place in the CPO reactor 10 can have a direct impact on the composition of the produced syngas in the CPO reactor effluent 15 which, according to this disclosure can comprise the hydrogen-lean syngas. In the absence of any side reaction (theoretically), the CPO reaction as represented by equation (1) results in a syngas with an H₂/CO molar ratio of 2.0. However, the presence of side reactions can (practically) reduce H₂ (and increase CO₂), thereby resulting in a syngas with a \molar ratio of H₂/CO that is not equal to 2.

Further, without wishing to be limited by theory, the CPO reaction as depicted in equation (1) is an exothermic heterogeneous catalytic reaction (i.e., a mildly exothermic reaction) and it occurs in a single reactor unit, such as the CPO reactor 10 (as opposed to more than one reactor unit as is the case in conventional processes for syngas production, such as steam methane reforming (SMR)—autothermal reforming (ATR) combinations). While it is possible to conduct partial oxidation of hydrocarbons as a homogeneous reaction, in the absence of a catalyst, homogeneous partial oxidation of hydrocarbons process entails excessive temperatures, long residence times, as well as excessive coke formation, which strongly reduce the controllability of the partial oxidation reaction, and may not produce syngas of the desired quality in a single reactor unit.

Furthermore, without wishing to be limited by theory, the CPO reaction is fairly resistant to chemical poisoning, and as such it allows for the use of a wide variety of hydrocarbon feedstocks, including some sulfur containing hydrocarbon feedstocks; which, in some cases, can enhance catalyst life-time and productivity. By contrast, conventional ATR processes have more restrictive feed requirements, for example in terms of content of impurities in the feed (e.g., feed to ATR is desulfurized), as well as hydrocarbon composition (e.g., ATR primarily uses a CH₄-rich feed).

In embodiments, the hydrocarbons suitable for use in a CPO reaction as disclosed herein can include methane, natural gas, natural gas liquids, liquefied petroleum gas (LPG), associated gas, well head gas, enriched gas, paraffins, shale gas, shale liquids, fluid catalytic cracking (FCC) off gas, refinery process gases, refinery off gases, stack gases, fuel gas from a fuel gas header, or combinations thereof. In embodiments, an amount of CO₂ and/or CO in the reactant mixture 5 can be increased by diluting a feed with gases (e.g., stack gases) containing CO₂ and/or CO. Such gases containing CO and/or CO₂ include, without limitation, stack gases, reducing gases, off gases rich in CO, such as used in the metal industry, crackers, and the like. For example, dedicated coking reactors can be utilized which, when injected with steam supply, air, and CO₂ deliver a continuous CO stream to CPO reactor 10.

In embodiments, reactant mixture 5 comprises fuel gases from a steam cracker and CPO reactor 10 is operated at a high CH₄/O₂ molar ratio by providing an autothermal mode of operation. In embodiments, a hydrogen content of the reactant mixture 5 can be adjusted to maintain an appropriate adiabatic rise.

The hydrocarbons can include any suitable hydrocarbons source, and can contain C₁-C₆ hydrocarbons, as well some heavier hydrocarbons. In embodiments, the CPO reactant mixture 5 can comprise natural gas. Generally, natural gas is composed primarily of methane, but can also contain ethane, propane and heavier hydrocarbons (e.g., iso-butane, n-butane, iso-pentane, n-pentane, hexanes, etc.), as well as very small quantities of nitrogen, oxygen, carbon dioxide, sulfur compounds, and/or water. The natural gas can be provided from a variety of sources including, but not limited to, gas fields, oil fields, coal fields, fracking of shale fields, biomass, landfill gas, and the like, or combinations thereof. In some embodiments, the CPO reactant mixture 5 can comprise primarily CH₄ and O₂, which can be introduced separately into CPO reactor 10, in embodiments.

The natural gas can comprise any suitable amount of methane. In some embodiments, the natural gas can comprise biogas. For example, the natural gas can comprise from about 45 mol % to about 80 mol % methane, from about 20 mol % to about 55 mol % carbon dioxide, and less than about 15 mol % nitrogen.

In embodiments, natural gas can comprise CH₄ in an amount of greater than or equal to about 45 mol %, about 50 mol %, about 55 mol %, about 60 mol %, about 65 mol %, about 70 mol %, about 75 mol %, about 80 mol %, about 82 mol %, about 84 mol %, about 86 mol %, about 88 mol %, about 90 mol %, about 91 mol %, about 92 mol %, about 93 mol %, about 94 mol %, about 95 mol %, about 96 mol %, or about 97 mol %.

According to this disclosure, the hydrocarbons in the reactant mixture 5 comprise greater than or equal to about 3, 4, 5, 6, 7, 8, 9, or 10 mol % of heavier hydrocarbons comprising hydrocarbons having two or more carbons (e.g., C2+ hydrocarbons). In embodiments, the hydrocarbons in the reactant mixture 5 comprise greater than or equal to about 3, 4, 5, 6, 7, 8, 9, or 10 mol % of C2+ alkanes. In embodiments, the hydrocarbons in reactant mixture 5 comprise ethane in an amount of greater than or equal to about 4, 5, 6, 7, 8, 9, 10, 11, 12, 13, 14, or 15 mol %. In embodiments, the hydrocarbons in the reactant mixture 5 comprise propane in an amount of greater than or equal to about 4, 5, 6, 7, 8, 9, 10, 11, 12, 13, 14, or 15 mol %. In embodiments, hydrocarbons comprise butanes in an amount of greater than or equal to about 3, 4, 5, 6, 7, or 8 mol %. In embodiments, the hydrocarbons in reactant mixture 5 comprise ethane in an amount of greater than or equal to about 4, 5, 6, 7, 8, 9, 10, 11, 12, 13, 14, or 15 mol %, propane in an amount of greater than or equal to about 4, 5, 6, 7, 8, 9, 10, 11, 12, 13, 14, or 15 mol %, butanes in an amount of greater than or equal to about 3, 4, 5, 6, 7, or 8 mol %, or a combination thereof.

In embodiments, the CPO reactant mixture 5 further comprises carbon dioxide (CO₂), and the CPO reactant mixture 5 is characterized by a CO₂ to carbon (CO₂/C) and/or a CO₂/CH₄ molar ratio in the CPO reactant mixture 5 of greater than or equal to about 0.5:1, 0.25:1, or 0:1, wherein the CO₂/C molar ratio refers to the total moles of CO₂ in the reactant mixture divided by the total moles of carbon (C) in the hydrocarbons in the reactant mixture 5. In embodiments, the CPO reactant mixture 5 further comprises carbon dioxide (CO₂), and the CPO reactant mixture 5 is characterized by a CO₂ to carbon (CO₂/C) molar ratio in the CPO reactant mixture 5 of less than or equal to about 10:1, 5:1, or 2:1. All or a portion of the CO₂ in reactant mixture 5 can be introduced into the reactant mixture 5 via CO₂ stream 7A, in embodiments. In embodiments, CPO reactor 10 is operated in autothermal mode with CO₂ injection or addition via 7A.

In embodiments, the amount of CO₂ in the CPO reactant mixture 5 is lower than the amount of CO₂ in a CPO reactant mixture in an otherwise similar process that produces a hydrogen-lean syngas from a reactant mixture comprising a lower quantity of C₂₊ alkanes hydrocarbons (e.g., wherein the hydrocarbons in the reactant mixture 5 comprise less than about 3 mol % C₂₊ alkanes). In embodiments, a portion of the carbon dioxide in the CPO reactor 10 undergoes a reverse water-gas shift (r-WGS) reaction within CPO reactor 10 (and/or in a r-WGS reactor 20 downstream of CPO reactor 10, as described hereinbelow), thereby decreasing the amount of hydrogen in the hydrogen-lean syngas.

In some embodiments, the hydrocarbons suitable for use in a CPO reaction as disclosed herein can comprise C₁-C₆ hydrocarbons (e.g., including C₂, C₃, and/or C₄ as described above), nitrogen (e.g., from about 0.1 mol % to about 15 mol %, alternatively from about 0.5 mol % to about 11 mol %, alternatively from about 1 mol % to about 7.5 mol %, or alternatively from about 1.3 mol % to about 5.5 mol %), and carbon dioxide (e.g., from about 0.1 mol % to about 2 mol %, alternatively from about 0.2 mol % to about 1 mol %, or alternatively from about 0.3 mol % to about 0.6 mol %). For example, the hydrocarbons suitable for use in a CPO reaction as disclosed herein can comprise C₁ hydrocarbon (about 89 mol % to about 92 mol %); C₂ hydrocarbons (greater than or equal to about 4, 5, 6, 7, 8, 9, 10, 11, 12, 13, 14, or 15 mol %); C₃ hydrocarbons (greater than or equal to about 4, 5, 6, 7, 8, 9, 10, 11, 12, 13, 14, or 15 mol %); C₄ hydrocarbons (greater than or equal to about 3, 4, 5, 6, 7, or 8 mol %); C₅ hydrocarbons (about 0.06 mol %); and C₆ hydrocarbons (about 0.02 mol %); and optionally nitrogen (about 0.1 mol % to about 15 mol %), carbon dioxide (about 0.1 mol % to about 2 mol %), or both nitrogen (about 0.1 mol % to about 15 mol %) and carbon dioxide (about 0.1 mol % to about 2 mol %).

The oxygen used in the CPO reactant mixture 5 can comprise 100% oxygen (substantially pure O₂), oxygen gas (which may be obtained via a membrane separation process), technical oxygen (which may contain some air), air, oxygen enriched air, oxygen-containing gaseous compounds (e.g., NO), oxygen-containing mixtures (e.g., O₂/CO₂, O₂/H₂O, O₂/H₂O₂/H₂O), oxy radical generators (e.g., CH₃OH, CH₂O), hydroxyl radical generators, and the like, or combinations thereof.

In embodiments, the CPO reactant mixture 5 can be characterized by a carbon to oxygen (C/O) or CH₄/O₂ molar ratio of less than about 3:1, alternatively less than about 2.6:1, alternatively less than about 2.4:1, alternatively less than about 2.2:1, alternatively less than about 2:1, alternatively less than about 1.8:1, alternatively greater than or equal to about 0.1:1, alternatively greater than or equal to about 0.2:1, alternatively greater than or equal to about 0.3:1, alternatively greater than or equal to about 0.4:1, alternatively greater than or equal to about 0.5:1, alternatively from about 0.5:1 to about 0.6:1, alternatively from about 0.55:1 to about 0.6:1, alternatively from about 0.5:1 to about 3:1, alternatively from about 0.7:1 to about 2.5:1, alternatively from about 0.9:1 to about 2.2:1, alternatively from about 1:1 to about 2:1, alternatively from about 1.5:1 to about 1.9:1, wherein the C/O molar ratio refers to the total moles of carbon (C) of hydrocarbons in the reactant mixture divided by the total moles of O₂ in the reactant mixture.

As the CPO reactant mixture 5 of this disclosure contains other carbon sources besides CH₄, such as ethane (C₂H₆), propane (C₃H₈), butanes (C₄H₁₀), etc., the C/O molar ratio accounts for the moles of carbon in each compound (e.g., 2 moles of C in 1 mole of C₂H₆, 3 moles of C in 1 mole of C₃H₈, 4 moles of C in 1 mole of C₄H₁₀, etc.). As will be appreciated by one of skill in the art, and with the help of this disclosure, the C/O molar ratio in the CPO reactant mixture 5 can be adjusted along with other reactor process parameters (e.g., temperature, pressure, flow velocity, etc.) to provide for a hydrogen-lean syngas as described herein. The C/O molar ratio in the CPO reactant mixture 5 can be adjusted to provide for a decreased amount of unconverted hydrocarbons in the syngas. The C/O molar ratio in the CPO reactant mixture 5 can be adjusted based on the CPO reactor temperature in order to decrease (e g, minimize) the unconverted hydrocarbons content of the CPO reactor effluent 15 comprising the hydrogen-lean syngas.

In embodiments, a CPO reactor suitable for use in the present disclosure (e.g., CPO reactor 10) can comprise a tubular reactor, a continuous flow reactor, a fixed bed reactor, a fluidized bed reactor, a moving bed reactor, a circulating fluidized bed reactor (e.g., a riser type reactor), a bubbling bed reactor, an ebullated bed reactor, a rotary kiln reactor, and the like, or combinations thereof. In some embodiments, the CPO reactor can comprise a circulating fluidized bed reactor, such as a riser type reactor.

In some embodiments, the CPO reactor 10 can be characterized by at least one CPO operational parameter selected from the group consisting of a CPO reactor temperature (e.g., CPO catalyst bed temperature); CPO feed temperature (e.g., temperature of CPO reactant mixture 5; target temperature of CPO reactor effluent 15; a CPO pressure (e.g., pressure of CPO reactor 10); a CPO contact time (e.g., CPO reactor 10 contact time); a C/O molar ratio in the CPO reactant mixture 5; a steam to carbon (S/C) molar ratio in the CPO reactant mixture 5, wherein the S/C molar ratio refers to the total moles of water (H₂O) in the reactant mixture 5 divided by the total moles of carbon (C) of hydrocarbons in the reactant mixture 5; and combinations thereof. For purposes of the disclosure herein, the CPO effluent temperature is the temperature of the syngas (e.g., hydrogen-lean syngas or CPO reactor effluent 15) measured at the point where the syngas exits the CPO reactor (e.g., CPO reactor 10), e.g., a temperature of the syngas measured at a CPO reactor outlet, a temperature of the syngas reactor effluent, a temperature of the exit syngas effluent. For purposes of the disclosure herein, the CPO effluent temperature (e.g., target CPO effluent temperature) is considered an operational parameter. As will be appreciated by one of skill in the art, and with the help of this disclosure, the choice of operational parameters for the CPO reactor such as CPO feed temperature; CPO pressure; CPO contact time; C/O molar ratio in the CPO reactant mixture; S/C molar ratio in the CPO reactant mixture; etc. determines the temperature of the syngas effluent (e.g., CPO reactor effluent 15), as well as the composition of the syngas effluent (e.g., CPO reactor effluent 15). Further, and as will be appreciated by one of skill in the art, and with the help of this disclosure, monitoring the CPO effluent temperature can provide feedback for changing other operational parameters (e.g., CPO feed temperature; CPO pressure; CPO contact time; C/O molar ratio in the CPO reactant mixture; S/C molar ratio in the CPO reactant mixture; etc.) as necessary for the CPO effluent temperature to match the target CPO effluent temperature. Furthermore, and as will be appreciated by one of skill in the art, and with the help of this disclosure, the target CPO effluent temperature is the desired CPO effluent temperature, and the CPO effluent temperature (e.g., measured CPO effluent temperature, actual CPO effluent temperature) may or may not coincide with the target CPO effluent temperature. In embodiments where the CPO effluent temperature is different from the target CPO effluent temperature, one or more CPO operational parameters (e.g., CPO feed temperature; CPO pressure; CPO contact time; C/O molar ratio in the CPO reactant mixture; S/C molar ratio in the CPO reactant mixture; etc.) can be adjusted (e.g., modified) in order for the CPO effluent temperature to match (e.g., be the same with, coincide with) the target CPO effluent temperature. The CPO reactor 10 can be operated under any suitable operational parameters as described herein that can provide for a hydrogen-lean syngas as described herein with a H₂/CO molar ratio in a range of from about 0.8 to 1.6, from about 0.8 to about 1.2, from about 0.9 to about 1.1, or equal to about 1.

The CPO reactor 10 can be characterized by a CPO reactant mixture temperature of from about 25° C. to about 600° C., alternatively from about 25° C. to about 500° C., alternatively from about 25° C. to about 400° C., alternatively from about 50° C. to about 400° C., alternatively from about 100° C. to about 400° C., or alternatively from about 100° C. to about 500° C. In embodiments, the CPO reactor 10 can be characterized by a CPO reactor temperature of less than 1200, 1100, or 1000° C.

The CPO reactor 10 can be characterized by a CPO effluent temperature (e.g., target CPO effluent 15 temperature) of greater than or equal to about 300° C., greater than or equal to about 600° C., alternatively greater than or equal to about 700° C., alternatively greater than or equal to about 750° C., alternatively greater than or equal to about 800° C., alternatively greater than or equal to about 850° C., alternatively from about 300° C. to about 1,600° C., alternatively from about 600° C. to about 1,400° C., alternatively from about 600° C. to about 1,300° C., alternatively from about 700° C. to about 1,200° C., alternatively from about 750° C. to about 1,150° C., alternatively from about 800° C. to about 1,125° C., or alternatively from about 850° C. to about 1,100° C.

In embodiments, the CPO reactor 10 can be characterized by any suitable reactor temperature and/or catalyst bed temperature. For example, the CPO reactor 10 can be characterized by a reactor temperature and/or catalyst bed temperature of greater than or equal to about 300° C., alternatively greater than or equal to about 600° C., alternatively greater than or equal to about 700° C., alternatively greater than or equal to about 750° C., alternatively greater than or equal to about 800° C., alternatively greater than or equal to about 850° C., alternatively from about 300° C. to about 1,600° C., alternatively from about 600° C. to about 1,400° C., alternatively from about 600° C. to about 1,300° C., alternatively from about 700° C. to about 1,200° C., alternatively from about 750° C. to about 1,150° C., alternatively from about 800° C. to about 1,125° C., or alternatively from about 850° C. to about 1,100° C.

The CPO reactor 10 can be operated under any suitable temperature profile that can provide for a hydrogen-lean syngas as described herein. The CPO reactor 10 can be operated under adiabatic conditions, non-adiabatic conditions, isothermal conditions, near-isothermal conditions, autothermal conditions, etc. For purposes of the disclosure herein, the term “non-adiabatic conditions” refers to process conditions wherein a reactor is subjected to external heat exchange or transfer (e.g., the reactor is heated; or the reactor is cooled), which can be direct heat exchange and/or indirect heat exchange. As will be appreciated by one of skill in the art, and with the help of this disclosure, the terms “direct heat exchange” and “indirect heat exchange” are known to one of skill in the art. By contrast, the term “adiabatic conditions” refers to process conditions wherein a reactor is not subjected to external heat exchange (e.g., the reactor is not heated; or the reactor is not cooled). Generally, external heat exchange implies an external heat exchange system (e.g., a cooling system; a heating system) that requires energy input and/or output. External heat transfer can also result from heat loss from the catalyst bed (or reactor) due to radiation, conduction or convection. For example, this heat exchange from the catalyst bed can be to the external environment or to the reactor zones before and after the catalyst bed.

For purposes of the disclosure herein, the term “isothermal conditions” refers to process conditions (e.g., CPO operational parameters) that allow for a substantially constant temperature of the reactor and/or catalyst bed (e.g., isothermal temperature) that can be defined as a temperature that varies by less than about ±10° C., alternatively less than about ±9° C., alternatively less than about ±8° C., alternatively less than about ±7° C., alternatively less than about ±6° C., alternatively less than about ±5° C., alternatively less than about ±4° C., alternatively less than about ±3° C., alternatively less than about ±2° C., or alternatively less than about ±1° C. across the reactor and/or catalyst bed, respectively. Further, for purposes of the disclosure herein, the term “isothermal conditions” comprise a temperature variation of less than about ±10° C. across the reactor and/or catalyst bed. In embodiments, the CPO reactor 10 can be operated under any suitable operational parameters that can provide for isothermal conditions.

For purposes of the disclosure herein, the term “near-isothermal conditions” refers to process conditions (e.g., CPO operational parameters) that allow for a fairly constant temperature of the reactor and/or catalyst bed (e.g., near-isothermal temperature), which can be defined as a temperature that varies by less than about ±100° C., alternatively less than about ±90° C., alternatively less than about ±80° C., alternatively less than about ±70° C., alternatively less than about ±60° C., alternatively less than about ±50° C., alternatively less than about ±40° C., alternatively less than about ±30° C., alternatively less than about ±20° C., alternatively less than about ±10° C., alternatively less than about ±9° C., alternatively less than about ±8° C., alternatively less than about ±7° C., alternatively less than about ±6° C., alternatively less than about ±5° C., alternatively less than about ±4° C., alternatively less than about ±3° C., alternatively less than about ±2° C., or alternatively less than about ±1° C. across the reactor and/or catalyst bed, respectively. In some embodiments, near-isothermal conditions allow for a temperature variation of less than about ±50° C., alternatively less than about ±25° C., or alternatively less than about ±10° C. across the reactor and/or catalyst bed. Further, for purposes of the disclosure herein, the term “near-isothermal conditions” is understood to include “isothermal” conditions.

Furthermore, for purposes of the disclosure herein, the term “near-isothermal conditions” refers to process conditions that comprise a temperature variation of less than about ±100° C. across the reactor and/or catalyst bed. In embodiments, a process as disclosed herein can comprise conducting the CPO reaction under near-isothermal conditions to produce the hydrogen-lean syngas, wherein the near-isothermal conditions comprise a temperature variation of less than about ±100° C. across the reactor and/or catalyst bed. In embodiments, the CPO reactor 10 can be operated under any suitable operational parameters that can provide for near-isothermal conditions.

Near-isothermal conditions can be provided by a variety of process and catalyst variables, such as temperature (e.g., heat exchange or heat transfer), pressure, gas flow rates, reactor configuration, catalyst bed configuration, catalyst bed composition, reactor cross sectional area, feed gas staging, feed gas injection, feed gas composition, and the like, or combinations thereof. Generally, and without wishing to be limited by theory, the terms “heat transfer” or “heat exchange” refer to thermal energy being exchanged or transferred between two systems (e.g., two reactors, such as a CPO reactor and a cracking reactor), and the terms “heat transfer” or “heat exchange” are used interchangeably for purposes of the disclosure herein.

In some embodiments, achieving a target CPO effluent temperature and/or near-isothermal conditions can be provided by heat exchange or heat transfer. The heat exchange can comprise heating the reactor; or cooling the reactor. In embodiments, achieving a target CPO effluent temperature and/or near-isothermal conditions can be provided by cooling the reactor. In another embodiment, achieving a target CPO effluent temperature and/or near-isothermal conditions can be provided by heating the reactor.

In some embodiments, achieving a target CPO effluent temperature and/or near-isothermal conditions can be provided by direct heat exchange and/or indirect heat exchange. As will be appreciated by one of skill in the art, and with the help of this disclosure, the terms “direct heat exchange” and “indirect heat exchange” are known to one of skill in the art.

The heat exchange can comprise external heat exchange, external coolant fluid cooling, reactive cooling, liquid nitrogen cooling, cryogenic cooling, electric heating, electric arc heating, microwave heating, radiant heating, natural gas combustion, solar heating, infrared heating, use of a diluent in the CPO reactant mixture, and the like, or combinations thereof. For example, reactive cooling can be effected by carrying out an endothermic reaction in a cooling coil/jacket associated with (e.g., located in) the reactor.

In some embodiments, achieving a target CPO effluent temperature and/or near-isothermal conditions can be provided by removal of process heat from the CPO reactor. In other embodiments, achieving a target CPO effluent temperature and/or near-isothermal conditions can be provided by supplying heat to the CPO reactor. As will be appreciated by one of skill in the art, and with the help of this disclosure, a CPO reactor may need to undergo both heating and cooling in order to achieve a target CPO effluent temperature and/or near-isothermal conditions.

In embodiments, the heat exchange or heat transfer can comprise introducing a cooling agent, such as a diluent, into the reactor (e.g., CPO reactor 10), to decrease the reactor temperature and/or the catalyst bed temperature, while increasing a temperature of the cooling agent and/or changing the phase of the cooling agent. The cooling agent can be reactive or non-reactive. The cooling agent can be in liquid state and/or in vapor state. As will be appreciated by one of skill in the art, and with the help of this disclosure, the cooling agent can act as a flammability retardant; for example by reducing the temperature inside the reactor, by changing the gas mixture composition, by reducing the combustion of hydrocarbons to carbon dioxide; etc.

In some embodiments, the CPO reactant mixture 5 can further comprise a diluent, wherein the diluent contributes to achieving a target CPO effluent temperature and/or near-isothermal conditions via heat exchange, as disclosed herein. The diluent can comprise water, steam, inert gases (e.g., argon), nitrogen, carbon dioxide, and the like, or combinations thereof. Generally, the diluent is inert with respect to the CPO reaction, e.g., the diluent does not participate in the CPO reaction. However, and as will be appreciated by one of skill in the art, and with the help of this disclosure, some diluents (e.g., water, steam, carbon dioxide, etc.) might undergo chemical reactions other than the CPO reaction within the reactor, and can change the composition of the resulting syngas, as will be described in more detail later herein; while other diluents (e.g., nitrogen (N₂), argon (Ar)) might not participate in reactions that change the composition of the resulting syngas. As will be appreciated by one of skill in the art, and with the help of this disclosure, the diluent can be used to vary the composition of the resulting syngas. The diluent can be present in the CPO reactant mixture 5 in any suitable amount.

The CPO reactor 10 can be characterized by a CPO pressure (e.g., reactor pressure measured at the reactor exit or outlet) of greater than or equal to about 1 barg, alternatively greater than or equal to about 10 barg, alternatively greater than or equal to about 20 barg, alternatively greater than or equal to about 25 barg, alternatively greater than or equal to about 30 barg, alternatively greater than or equal to about 35 barg, alternatively greater than or equal to about 40 barg, alternatively greater than or equal to about 50 barg, alternatively less than about 30 barg, alternatively less than about 25 barg, alternatively less than about 20 barg, alternatively less than about 10 barg, alternatively from about 1 barg to about 90 barg, alternatively from about 1 barg to about 70 barg, alternatively from about 1 barg to about 40 barg, alternatively from about 1 barg to about 30 barg, alternatively from about 1 barg to about 25 barg, alternatively from about 1 barg to about 20 barg, alternatively from about 1 barg to about 10 barg, alternatively from about 20 barg to about 90 barg, alternatively from about 25 barg to about 85 barg, or alternatively from about 20 barg to about 60 barg.

The CPO reactor 10 can be characterized by a CPO contact time of from about 0.001 milliseconds (ms) to about 5 seconds (s), alternatively from about 0.001 ms to about 1 s, alternatively from about 0.001 ms to about 100 ms, alternatively from about 0.001 ms to about 10 ms, alternatively from about 0.001 ms to about 5 ms, or alternatively from about 0.01 ms to about 1.2 ms. Generally, the contact time of a reactor comprising a catalyst refers to the average amount of time that a compound (e.g., a molecule of that compound) spends in contact with the catalyst (e.g., within the catalyst bed), e.g., the average amount of time that it takes for a compound (e.g., a molecule of that compound) to travel through the catalyst bed. In some embodiments, the CPO reactor 10 can be characterized by a contact time of from about 0.001 ms to about 5 ms, or alternatively from about 0.01 ms to about 1.2 ms.

All of the CPO operational parameters disclosed herein are applicable throughout all of the embodiments disclosed herein, unless otherwise specified. As will be appreciated by one of skill in the art, and with the help of this disclosure, each CPO operational parameter can be adjusted to provide for a hydrogen-lean syngas as described herein. For example, the CPO operational parameters can be adjusted to provide for an increased H₂ content of the syngas, so long as the H₂/CO molar ratio remains in the desired range (e.g., from about 0.8 to about 1.6). As another example, the CPO operational parameters can be adjusted to provide for a decreased CO₂ content of the syngas in the CPO reactor effluent 15. As yet another example, the CPO operational parameters can be adjusted to provide for a decreased unreacted hydrocarbons (e.g., unreacted CH₄) content of the syngas in the CPO reactor effluent 15.

In embodiments, the CPO reactor 10 is characterized by at least one CPO operational parameter selected from the group consisting of a CPO reactant temperature of from about 100° C. to about 500° C.; a CPO pressure of from about 20 barg to about 80 barg; a CPO contact time of from about 0.001 milliseconds (ms) to about 5 seconds (s); a carbon to oxygen (C/O) molar ratio in the CPO reactant mixture of from about 0.5:1 to about 3:1, wherein the C/O molar ratio refers to the total moles of carbon (C) in the hydrocarbons in the reactant mixture divided by the total moles of oxygen (O₂) in the reactant mixture; a steam to carbon (S/C) molar ratio in the CPO reactant mixture of less than about 0.6:1, wherein the S/C molar ratio refers to the total moles of water (H₂O) in the reactant mixture divided by the total moles of carbon (C) in the hydrocarbons in the reactant mixture; and combinations thereof.

In embodiments, the CPO reactor 10 is characterized by at least one CPO operational parameter selected from the group consisting of a CPO reactant mixture temperature of from about 100° C. to about 500° C.; a CPO pressure of from about 25 barg to about 80 barg; a CPO contact time of from about 0.001 milliseconds (ms) to about 5 seconds (s); a carbon to oxygen (C/O) molar ratio in the CPO reactant mixture of from about 0.5:1 to about 2:1, wherein the C/O molar ratio refers to the total moles of carbon (C) in the hydrocarbons in the reactant mixture divided by the total moles of oxygen (O₂) in the reactant mixture; a steam to carbon (S/C) molar ratio in the CPO reactant mixture of less than about 0.25:1, wherein the S/C molar ratio refers to the total moles of water (H₂O) in the reactant mixture divided by the total moles of carbon (C) in the hydrocarbons in the reactant mixture; and combinations thereof.

The CPO reaction is an exothermic reaction (e.g., heterogeneous catalytic reaction; exothermic heterogeneous catalytic reaction) that is generally conducted in the presence of a CPO catalyst comprising a catalytically active metal, i.e., a metal active for catalyzing the CPO reaction. The catalytically active metal can comprise a noble metal (e.g., Pt, Rh, Ir, Pd, Ru, Ag, and the like, or combinations thereof); a non-noble metal (e.g., Ni, Co, V, Mo, P, Fe, Cu, and the like, or combinations thereof); rare earth elements (e.g., La, Ce, Nd, Eu, and the like, or combinations thereof); oxides thereof; and the like; or combinations thereof. Generally, a noble metal is a metal that resists corrosion and oxidation in a water-containing environment. As will be appreciated by one of skill in the art, and with the help of this disclosure, the components of the CPO catalyst (e.g., metals such as noble metals, non-noble metals, rare earth elements) can be either phase segregated or combined within the same phase.

In embodiments, the CPO catalysts suitable for use in the present disclosure can be supported catalysts and/or unsupported catalysts. In some embodiments, the supported catalysts can comprise a support, wherein the support can be catalytically active (e.g., the support can catalyze a CPO reaction). For example, the catalytically active support can comprise a metal gauze or wire mesh (e.g., Pt gauze or wire mesh); a catalytically active metal monolithic catalyst; etc. In other embodiments, the supported catalysts can comprise a support, wherein the support can be catalytically inactive (e.g., the support cannot catalyze a CPO reaction), such as SiO₂; silicon carbide (SiC); alumina; a catalytically inactive monolithic support; etc. In yet other embodiments, the supported catalysts can comprise a catalytically active support and a catalytically inactive support.

In some embodiments, a CPO catalyst can be wash coated onto a support, wherein the support can be catalytically active or inactive, and wherein the support can be a monolith, a foam, an irregular catalyst particle, etc.

In some embodiments, the CPO catalyst can be a monolith, a foam, a powder, a particle, etc. Nonlimiting examples of CPO catalyst particle shapes suitable for use in the present disclosure include cylindrical, discoidal, spherical, tabular, ellipsoidal, equant, irregular, cubic, acicular, and the like, or combinations thereof.

In some embodiments, the support comprises an inorganic oxide, alpha, beta or theta alumina (Al₂O₃), activated Al₂O₃, silicon dioxide (SiO₂), titanium dioxide (TiO₂), magnesium oxide (MgO), zirconium oxide (ZrO₂), lanthanum (III) oxide (La₂O₃), yttrium (III) oxide (Y₂O₃), cerium (IV) oxide (CeO₂), zeolites, ZSM-5, perovskite oxides, hydrotalcite oxides, and the like, or combinations thereof.

Without limitation, CPO processes, CPO reactors, CPO catalysts, and CPO catalyst bed configurations suitable for use in the present disclosure are described in more detail in U.S. Provisional Patent Application No. 62/522,910 filed Jun. 21, 2017 (International Application No. PCT/IB2018/054475 filed Jun. 18, 2018) and entitled “Improved Reactor Designs for Heterogeneous Catalytic Reactions;” and U.S. Provisional Patent Application No. 62/521,831 filed Jun. 19, 2017 (International Application No. PCT/IB2018/054470 filed Jun. 18, 2018) and entitled “An Improved Process for Syngas Production for Petrochemical Applications;” each of which is hereby incorporated herein by reference in its entirety for purposes not contrary to this disclosure.

In embodiments, the CPO catalyst can be characterized by a catalyst productivity variation within about +20%, alternatively within about +17.5%, alternatively within about +15%, alternatively within about +12.5%, alternatively within about +10%, alternatively within about +7.5%, alternatively within about +5%, alternatively within about +2.5%, or alternatively within about +1% of a target catalyst productivity over a time period of equal to or greater than about 500 hours (h), alternatively equal to or greater than about 1,000 h, alternatively equal to or greater than about 2,500 h, alternatively equal to or greater than about 5,000 h, alternatively equal to or greater than about 7,500 h, or alternatively equal to or greater than about 10,000 h; wherein catalyst productivity is defined as the amount of syngas in CPO reactor effluent 15 recovered from the CPO reactor 10 divided by the amount of hydrocarbons introduced to the CPO reactor 10 in CPO reactant mixture 5. As will be appreciated by one of skill in the art, and with the help of this disclosure, and without wishing to be limited by theory, catalyst productivity is a quantitative measure of catalyst activity, wherein the catalyst activity refers to the ability of a catalyst (e.g., CPO catalyst) to increase the rate of a chemical reaction (e.g., CPO reaction) under a given set of reaction conditions (e.g., CPO operational parameters). For purposes of the disclosure herein, a CPO catalyst having a productivity variation greater than about +20% can be referred to as a “spent CPO catalyst” (as opposed to an active CPO catalyst). As used herein, the target catalyst productivity is associated with an active CPO catalyst (e.g., fresh CPO catalyst and/or regenerated CPO catalyst). For purposes of the disclosure herein, the term “fresh CPO catalyst” refers to a CPO catalyst that has not been used in a CPO process. As will be appreciated by one of skill in the art, and with the help of this disclosure, an active CPO catalyst displays optimum (e.g., maximum) catalyst activity with respect to a chemical reaction (e.g., CPO reaction) under a given set of reaction conditions (e.g., CPO operational parameters). Further, and as will be appreciated by one of skill in the art, and with the help of this disclosure, the target catalyst productivity is the maximum catalyst productivity of an active CPO catalyst (e.g., fresh CPO catalyst and/or regenerated CPO catalyst) under a given set of reaction conditions (e.g., CPO operational parameters). Furthermore, and as will be appreciated by one of skill in the art, and with the help of this disclosure, the terms “catalyst productivity” and “target catalyst productivity” are used in the context of steady-state operation of the CPO reactor (e.g., CPO reactor 10).

As will be appreciated by one of skill in the art, and with the help of this disclosure, catalyst activity (e.g., CPO catalyst activity) can vary (e.g., decay, decrease) over time, for a variety of reasons, such as poisoning (e.g., feed contaminants), fouling (e.g., coking by carbon produced by cracking/condensation/decomposition reactions of hydrocarbon reactants, intermediates, and/or products), thermal degradation (e.g., collapse of support structure, solid-state reactions, attrition), active component leaching, migration of active components within and/or outside catalyst particles, side reactions, attrition/crushing, and the like, or combinations thereof. Decay in catalyst activity leads to spent catalysts (e.g., spent CPO catalysts). In embodiments, spent catalysts can be regenerated and returned to a production process, as will be described in more detail later herein.

In embodiments, a portion of the hydrocarbons (e.g., methane) in the CPO reactant mixture 5 can undergo a thermal decomposition reaction to carbon (C) and H₂, for example as represented by eqn. (2):

CH₄→C+2H₂ (2)

The decomposition reaction of hydrocarbons, such as methane, is facilitated by elevated temperatures, and increases the hydrogen content in the syngas in CPO reactor effluent 15. However, the carbon produced by the decomposition reaction of hydrocarbons (e.g., a decomposition reaction as represented by equation (2)) can lead to coking of the CPO catalyst via carbon deposition onto the CPO catalyst, thereby producing a spent CPO catalyst. As will be appreciated by one of skill in the art, and with the help of this disclosure, and without wishing to be limited by theory, while the percentage of hydrocarbons in the CPO reactant mixture 5 that undergoes a decomposition reaction (e.g., a decomposition reaction as represented by equation (2)) increases with increasing the C/O molar ratio in the CPO reactant mixture 5, a portion of hydrocarbons can undergo a decomposition reaction to C and H₂ even at relatively low C/O molar ratios in the CPO reactant mixture 5 (e.g., a C/O molar ratio in the CPO reactant mixture 5 of less than about 1:1). Further, and as will be appreciated by one of skill in the art, and with the help of this disclosure, the quality of the hydrocarbon feed to the CPO reactor 10 can influence coking. For example, higher hydrocarbons (e.g., hydrocarbons having equal to or greater than 2 C atoms, C₂₊) can produce more coke than methane, owing to having a higher carbon content than methane.

In an aspect, the CPO reactant mixture 5 can further comprise a diluent, such as water and/or steam, and CO₂. The CPO reactor 10 can be operated under any suitable operational conditions (e.g., CPO operational parameters) that can provide for a syngas with a desired composition (e.g., a desired H₂/CO molar ratio; a desired CO₂ content; etc.); for example, the CPO reactor 10 can be operated with introducing water and/or steam, and CO₂ to the CPO reactor 10.

When carbon is present in the reactor (e.g., coke; C produced as a result of a decomposition reaction as represented by equation (2)), water and/or steam diluent can react with the carbon and generate additional CO and H₂, for example as represented by equation (3):

C+H₂O [symbol] CO+H₂ (3)

As will be appreciated by one of skill in the art, and with the help of this disclosure, the presence of water and/or steam in the CPO reactor 10 can decrease the amount of coke in the CPO reactor 10 (e.g., the amount of coke deposited on the CPO catalyst, the amount of spent CPO catalyst present in the CPO reactor 10), thereby providing for maintaining the catalyst productivity.

Further, and as will be appreciated by one of skill in the art, and with the help of this disclosure, water and/or steam can be used to vary the composition of the resulting syngas in CPO reactor effluent 15. Steam can react with methane, for example as represented by equation (4):

CH₄+H₂O [symbol] CO+3H₂ (4)

In an aspect, a diluent comprising water and/or steam can increase a hydrogen content of the resulting syngas in CPO reactor effluent 15. For example, in aspects where the CPO reactant mixture 5 comprises water and/or steam diluent, the resulting syngas in CPO reactor effluent 15 can be characterized by a hydrogen to carbon monoxide molar ratio that is increased when compared to a hydrogen to carbon monoxide molar ratio of a syngas produced by an otherwise similar process conducted with a reactant mixture comprising hydrocarbons and oxygen without the water and/or steam diluent. Without wishing to be limited by theory, the reforming reaction (e.g., as represented by equation (4)) is an endothermic reaction. The reforming reaction as represented by equation (4) can remove a portion of the process heat (e.g., heat produced by the exothermic CPO reaction, for example as represented by equation (1)).

In the presence of water and/or steam in the CPO reactor 10, carbon monoxide can react with the water and/or steam to form carbon dioxide and hydrogen via a water-gas shift (WGS) reaction, for example as represented by equation (5):

CO+H₂O [symbol] CO₂+H₂ (5)

While the WGS reaction can increase the H₂/CO molar ratio of the syngas produced by the CPO reactor 10, it also produces CO₂.

Injection of steam and/or water can help maintain CPO catalyst activity. In embodiments, the CPO reactant mixture 5 can be characterized by a steam to carbon (S/C) and/or a steam to CH₄ (S/CH₄) molar ratio of less than or equal to 1.0, 0.5, 0.4, 0.3, 0.2, or in a range of from about 0.1 to 01.0, 0.2 to 0.6, or 0.2 to 0.5. In embodiments, the CPO reactor 10 can be operated at an S/C molar ratio) and/or a steam to CH₄ (S/CH₄) molar ratio in the CPO reactant mixture 5 of less than about 0.6:1, alternatively less than about 0.5:1, alternatively less than about 0.4:1, alternatively less than about 0.3:1, alternatively less than about 0.2:1, alternatively less than about 0.1:1, alternatively from about 0.01:1 to less than about 0.6:1, alternatively from about 0.05:1 to about 0.6:1, alternatively from about 0.1:1 to about 0.5:1, alternatively from about 0.15:1 to about 0.6:1, or alternatively from about 0.2:1 to about 0.6:1. As will be appreciated by one of skill in the art, and with the help of this disclosure, the steam that is introduced to the CPO reactor for use as a diluent in a CPO reaction as disclosed herein is present in significantly smaller amounts than the amounts of steam utilized in steam reforming (e.g., SMR) processes, and as such, a process for producing syngas as disclosed herein can yield a (e.g., hydrogen-lean) syngas with lower amounts of hydrogen when compared to the amounts of hydrogen in a syngas produced by steam reforming.

The S/C molar ratio in the CPO reactant mixture 10 can be adjusted based on the desired CPO effluent temperature (e.g., target CPO effluent temperature) in order to adjust the H₂ content of the produced syngas (e.g., syngas 15). As will be appreciated by one of skill in the art, and with the help of this disclosure, the reaction (4) that consumes steam in the CPO reactor may be less preferable over the water-gas shift (WGS) reaction (5) in the CPO reactor 10, as reaction (4) allows for increasing the H₂ content of the produced syngas (e.g., syngas 15), as well as the M ratio of the produced syngas (e.g., syngas 15), wherein the M ratio is a molar ratio defined as (H₂—CO₂)/(CO+CO₂). Further, and as will be appreciated by one of skill in the art, and with the help of this disclosure, reaction (5) converts water and CO to both H₂ and CO₂.

Without wishing to be limited by theory, the presence of water and/or steam in the CPO reactor 10 changes the flammability of the CPO reactant mixture 10, thereby providing for a wider practical range of C/O molar ratios in the CPO reactant mixture 10. Further, and without wishing to be limited by theory, the presence of water and/or steam in the CPO reactor 10 allows for the use of lower C/O molar ratios in the CPO reactant mixture 10. Furthermore, and without wishing to be limited by theory, the presence of water and/or steam in the CPO reactor 10 allows for operating the CPO reactor 10 at relatively high pressures.

As will be appreciated by one of skill in the art, and with the help of this disclosure, the introduction of water and/or steam in the CPO reactor 10 can lead to increasing the amount of unreacted hydrocarbons in the syngas 15. Further, as will be appreciated by one of skill in the art, and with the help of this disclosure, some downstream chemical synthesis processes tolerate limited amounts of unreacted hydrocarbons in the syngas.

In some aspects, the syngas 15 can comprise less than about 7.5 mol %, alternatively less than about 5 mol %, or alternatively less than about 2.5 mol % hydrocarbons (e.g., unreacted hydrocarbons, unreacted CH₄). In such aspects, the syngas 15 can be produced in a CPO process that employs water and/or steam.

In embodiments, CO₂ is introduced into the CPO reactor 10 (e.g., via line 7A). Since oxygen is present in the CPO reactant mixture 5, the carbon present in the reactor (e.g., coke; C produced as a result of a decomposition reaction as represented by equation (2)) can also react with oxygen, for example as represented by equation (6):

C+O₂→CO₂ (6)

When carbon is present in the reactor (e.g., coke; C produced as a result of a decomposition reaction as represented by equation (2)), CO₂ (e.g., introduced to the CPO reactor 10 as part of the CPO reactant mixture 5 and/or produced by the reaction represented by equation (6)) can react with the carbon, for example as represented by equation (7):

C+CO₂ [symbol] 2CO (7)

thereby decreasing the amount of CO₂ and increasing the amount of CO in the resulting syngas in the CPO reactor effluent 15. The use of reactant mixtures 5 comprising higher hydrocarbons (e.g., C2+) can lead to the formation of a greater amount of coke, and thus lead to an enrichment of CO and a reduced H₂/CO molar ratio in the syngas in CPO reactor effluent 15. As will be appreciated by one of skill in the art, and with the help of this disclosure, the presence of CO₂ in the CPO reactor 10 can decrease the amount of coke in the CPO reactor 10 (e.g., the amount of coke deposited on the CPO catalyst, the amount of spent CPO catalyst present in the CPO reactor 10), thereby providing for maintaining the catalyst productivity. Injection of CO₂ also provides for an enhancement in carbon efficiency, because the carbon in the CO₂ is converted to additional CO. As a result, more CO will be produced per MMBTU of reactant feed (e.g., natural gas) according to embodiments of this disclosure. This additional CO can contribute to an increase in chemical product throughput (e.g., from downstream synthesis 30) at the same flowrate of reactant feed (e.g., natural gas).

Furthermore, CO₂ can react with methane in a dry reforming reaction, for example as represented by equation (8):

CH₄+CO₂ [symbol] 2CO+2H₂ (8)

thereby decreasing the amount of CO₂ in the resulting syngas in CPO reactor effluent 15. Without wishing to be limited by theory, the dry reforming reaction (e.g., as represented by eqn. (8)) is an endothermic reaction (e.g., highly endothermic reaction). The dry reforming reaction can remove a portion of the process heat (e.g., heat produced by the exothermic CPO reaction, for example as represented by eqn. (1)).

In embodiments, a diluent comprising carbon dioxide can increase a carbon monoxide content of the resulting syngas in the CPO reactor effluent 15. For example, in embodiments where the CPO reactant mixture 5 comprises carbon dioxide, the syngas in CPO reactor effluent 15 can be characterized by a hydrogen to carbon monoxide molar ratio that is decreased when compared to a hydrogen to carbon monoxide molar ratio of a syngas produced by an otherwise similar process conducted with a reactant mixture comprising hydrocarbons and oxygen without the carbon dioxide diluent. Without wishing to be limited by theory, carbon dioxide can react with coke inside the CPO reactor 10 and generate additional CO, for example as represented by equation (7). Further, and without wishing to be limited by theory, carbon dioxide can participate in a dry reforming of methane reaction, thereby generating additional CO and H₂, for example as represented by equation (8). Dry reforming of methane is generally accompanied by a reaction between carbon dioxide and hydrogen which results in the formation of additional CO and water.

In embodiments, the CPO reactant mixture 5 can comprise carbon dioxide in an amount effective to provide for less than about 7 mol %, alternatively less than about 6 mol %, alternatively less than about 5 mol %, alternatively from about 0.1 mol % to about 7 mol %, alternatively from about 0.25 mol % to about 6 mol %, or alternatively from about 0.5 mol % to about 5 mol % carbon dioxide in the syngas in CPO reactor effluent 15, based on the total mol % of the syngas. The carbon dioxide of the CPO reactant mixture 5 can be CO₂ from natural gas sources, wherein the CO₂ is introduced to the CPO reactor 10 with the hydrocarbons; and/or additional or supplemental CO₂, for example CO₂ recovered as a process stream and recycled to the CPO reactor 10 (e.g., CO₂ stream 7A).

In embodiments, the conversion of hydrocarbons in the CPO reactor 10 is greater than the conversion of hydrocarbons in a CPO reactor in an otherwise similar process that produces a hydrogen-lean syngas from hydrocarbons comprising a reduced amount of higher hydrocarbons (e.g., C₂₊ hydrocarbons). For example, in embodiments, the conversion of hydrocarbons in the CPO reactor 10 of a reactant feed mixture 5 comprising greater than or equal to about 5, 4, or 3 mol % C₂₊ alkanes is greater than the conversion of hydrocarbons in a CPO reactor in an otherwise similar process that produces a hydrogen-lean syngas from a reactant mixture 5 comprising less than about 5, 4, or 3 mol % C₂₊ alkanes, respectively.

In embodiments, CPO reactor effluent 15 comprises the hydrogen-lean syngas, and no further adjustment of the molar ratio of H₂/CO is provided prior to downstream synthesis reactor of downstream synthesis apparatus 30. In embodiments, a process as disclosed herein can further comprise: (i) recovering a CPO reactor effluent 15 from the CPO reactor 10, wherein the CPO reactor effluent 15 comprises hydrogen, carbon monoxide, carbon dioxide, water, and unreacted hydrocarbons, and wherein the CPO reactor effluent 15 is characterized by a H₂/CO molar ratio of greater than about 1.6, 1.5, 1.4, 1.3, or 1.2; and (ii) feeding at least a portion of the CPO reactor effluent 15 to a reverse water-gas shift (r-WGS) reactor 20, wherein a portion of the hydrogen of the CPO reactor effluent 15 reacts with carbon dioxide via a r-WGS reaction to produce water and carbon monoxide, to produce the hydrogen-lean syngas, which can be removed from the r-WGS reactor 20 via r-WGS reactor effluent 25.

The r-WGS reaction is depicted in Equation (9):

CO₂+H₂↔CO+H₂O (9)

In embodiments, a process as disclosed herein can further comprise introducing additional carbon dioxide 7B to the r-WGS reactor 20 to drive the r-WGS reaction toward the production of carbon monoxide (i.e., to drive the WGS reaction of Equation 5 toward the production of carbon monoxide).

In embodiments, a process as disclosed herein can further comprise: (a) recovering a r-WGS reactor effluent 25 from the r-WGS reactor, wherein the r-WGS reactor effluent comprises hydrogen, carbon monoxide, carbon dioxide, water, and unreacted hydrocarbons; and (b) removing at least a portion of the water from the r-WGS reactor effluent 25 to yield the hydrogen-lean syngas, wherein the amount of water in the r-WGS reactor effluent is greater than the amount of water in the hydrogen-lean syngas.

In embodiments, a process as disclosed herein further comprises: (1) contacting a portion of the CPO reactor effluent 15 with at least a portion of the r-WGS reactor effluent 25 to produce a combined effluent stream; and (2) removing at least a portion of the water from the combined effluent stream to yield the hydrogen-lean syngas, wherein the amount of water in the combined effluent stream is greater than the amount of water in the hydrogen-lean syngas.

In embodiments, a process as disclosed herein excludes a step of introducing at least a portion of the CPO reactor effluent 15 and/or at least a portion of the hydrogen-lean syngas in r-WGS reactor effluent 25 to a hydrogen recovery unit to decrease the amount of hydrogen in the CPO reactor effluent 15 and/or the hydrogen-lean syngas in r-WGS reactor effluent 25, respectively.

In embodiments, the hydrogen-lean syngas (e.g., in the CPO reactor effluent 15 and/or the r-WGS reactor effluent 25) can have a CO₂ content of less than about 10 mol %, less than about 9 mol %, less than about 8 mol %, less than about 7 mol %, alternatively less than about 6 mol %, alternatively less than about 5 mol %, alternatively less than about 4 mol %, alternatively less than about 3 mol %, alternatively less than about 2 mol %, alternatively less than about 1 mol %, alternatively greater than about 0.1 mol %, alternatively greater than about 0.25 mol %, alternatively greater than about 0.5 mol %, alternatively from about 0.1 mol % to about 7 mol %, alternatively from about 0.25 mol % to about 6 mol %, or alternatively from about 0.5 mol % to about 5 mol %. As noted hereinabove, the CO₂ concentration in the hydrogen-lean syngas (e.g., in CPO reactor effluent 15 and/or r-WGS reactor effluent 25) can be controlled via CO₂ injection (e.g., via CO₂ 7A and/or 7B, respectively) and/or by changing the operating conditions of CPO reactor 10. The amount of CO₂ in the hydrogen-lean syngas can be adjusted depending on the downstream synthesis 30. For example, when small amounts of CO₂ are desirable in the hydrogen-lean syngas feed to downstream synthesis apparatus 30 (e.g., for downstream synthesis of DME, wherein small amounts of CO₂ in the hydrogen-lean syngas feed are desirable to increase the production of methanol intermediate and thus enhance DME synthesis), the CO₂ amount in the hydrogen-lean synthesis gas can be adjusted as provided hereinabove.

In embodiments, the CPO reactor effluent 15 and/or the r-WGS reactor effluent 25 can be subjected to processing, such as the recovery of unreacted hydrocarbons, diluent, water, etc. In embodiments, water can be condensed and separated from the CPO reactor effluent 15 and/or the r-WGS reactor effluent 25, e.g., in a condenser. As understood, such processing for the removal of hydrocarbons, diluent, water, etc. will not alter an H₂/CO molar ratio of the stream. In embodiments, a process as disclosed herein can further comprise (i) recovering at least a portion of the unreacted hydrocarbons from the CPO reactor effluent 15 and/or the r-WGS reactor effluent 25 to yield recovered hydrocarbons, and (ii) recycling at least a portion of the recovered hydrocarbons to the CPO reactor 10. As will be appreciated by one of skill in the art, and with the help of this disclosure, although fairly high conversions can be achieved in CPO processes (e.g., conversions of greater than or equal to about 90%), the unconverted hydrocarbons could be recovered and recycled back to the CPO reactor 10.

In embodiments, a CPO reactor with CO₂ injection as described hereinabove is utilized to produce the hydrogen-lean syngas for downstream chemical synthesis.

In embodiments, a process as disclosed herein further comprises using at least a portion of the hydrogen-lean syngas (e.g., in CPO reactor effluent 15 and/or r-WGS reactor effluent 25) in a downstream synthesis process comprising downstream synthesis apparatus 30.

The downstream synthesis process can be any process for which a hydrogen-lean syngas is utilized to produce at least one chemical product 35. For example, in embodiments and without limitation, in embodiments, the downstream process is selected from the group consisting of acetic acid synthesis process; dimethyl ether synthesis process; oxo-synthesis of aliphatic aldehydes and/or alcohols; and combinations thereof, and downstream synthesis apparatus 30 comprise apparatus operable for the synthesis of acetic acid 35A, the synthesis of dimethyl ether (DME) 35B, oxo-synthesis of aliphatic aldehydes 35C and/or alcohols 35D, or a combination thereof.

In embodiments, a process as disclosed herein does not comprise altering the H₂/CO molar ratio of the hydrogen-lean syngas (e.g., the CPO reactor effluent 15 and/or the r-WGS reactor effluent 25) between the CPO reactor 10 and a downstream synthesis reactor of the downstream synthesis apparatus 30. Thus, in embodiments, a chemical synthesis system as disclosed herein does not comprise apparatus (e.g., a hydrogen removal unit, PSA) for altering the H₂/CO molar ratio of the hydrogen-lean syngas (e.g., the CPO reactor effluent 15 and/or the r-WGS reactor effluent 25) between the CPO reactor 10 and the downstream synthesis reactor of downstream synthesis apparatus 30. In embodiments, a chemical synthesis system as disclosed herein comprises a reduced size apparatus (relative to conventional) for altering the H₂/CO molar ratio of the hydrogen-lean syngas (e.g., the CPO reactor effluent 15 and/or the r-WGS reactor effluent 25) between the CPO reactor 10 and the downstream synthesis reactor of downstream synthesis apparatus 30.

In embodiments, a process as disclosed herein comprises no adjusting of the H₂/CO molar ratio of the hydrogen-lean syngas other than optionally subjecting the CPO reactor effluent 15 to reverse water gas shift prior to the utilizing the hydrogen-lean syngas in a downstream chemical synthesis reactor of downstream chemical synthesis apparatus 30. Thus, in embodiments, a chemical synthesis system as disclosed herein comprises no apparatus for adjusting the H₂/CO molar ratio of the hydrogen-lean syngas other than an optional reverse water gas shift apparatus prior to a downstream synthesis reactor of the downstream synthesis apparatus 30.

In embodiments, a process as disclosed herein does not comprise removing a hydrogen stream from the hydrogen-lean syngas (e.g., the CPO reactor effluent 15 and/or the r-WGS reactor effluent 25) prior to utilizing the hydrogen-lean syngas in downstream chemical synthesis. Thus, in embodiments, a chemical synthesis system as disclosed herein comprises no apparatus configured to remove a hydrogen stream from the hydrogen-lean syngas between the CPO reactor and a downstream synthesis reactor of the downstream synthesis apparatus 30. In embodiments, a chemical synthesis system as disclosed herein comprises a reduced size apparatus (relative to conventional) configured to remove a hydrogen stream from the hydrogen-lean syngas between the CPO reactor and a downstream synthesis reactor of the downstream synthesis apparatus 30.

In embodiments, the CPO reactor 10 can produce the hydrogen lean syngas at high pressures (e.g., greater than or equal to about 20, 25, 30, 35, 40, 45, 50 bar) that are required for downstream chemical (e.g., acetic acid, DME) synthesis. Accordingly, the herein disclosed system and method for producing hydrogen-lean syngas via CPO can, in embodiments, further reduce energy requirements for production of the chemical produced in downstream synthesis 30.

In embodiments, a process as disclosed herein can advantageously display improvements in one or more process characteristics when compared to conventional processes.

As will be appreciated by one of skill in the art, and with the help of this disclosure, since the CPO reaction is exothermic, very little heat supply in the form of fuel combustion is needed (e.g., for pre-heating reactants in the reaction mixture 5 that is supplied to the CPO syngas generation section), when compared to conventional steam reforming. As such, the process for chemical synthesis utilizing CPO hydrogen-lean syngas as disclosed herein can advantageously generate less CO₂ through fuel burning, when compared to steam reforming.

The use of CPO reactant mixtures comprising higher hydrocarbons and/or CO₂ as described herein provides a high selectivity and thus increases the overall carbon efficiency of hydrogen-lean syngas synthesis relative to conventional processes. Because CPO can be operated at higher pressures than conventional syngas syntheses (e.g., dry reforming) utilized to produce hydrogen-lean syngas, compression requirements of the hydrogen-lean syngas prior to downstream chemical synthesis therefrom can be reduced (and/or such compression eliminated) relative to the conventional processes.

Additional advantages of the processes for the production of chemicals as disclosed herein can be apparent to one of skill in the art viewing this disclosure.

### Examples

The embodiments having been generally described, the following examples are given as particular embodiments of the disclosure and to demonstrate the practice and advantages thereof. It is understood that the examples are given by way of illustration and are not intended to limit the specification or the claims in any manner.

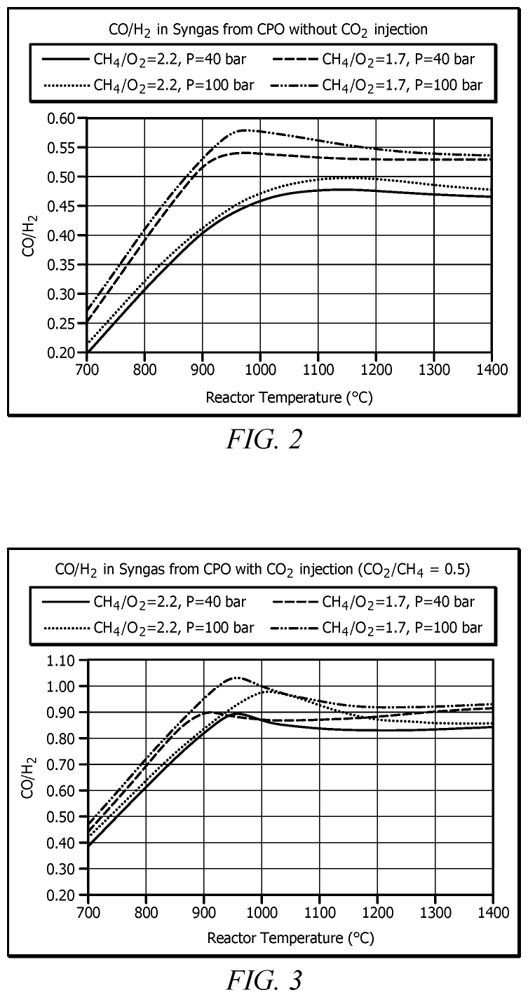

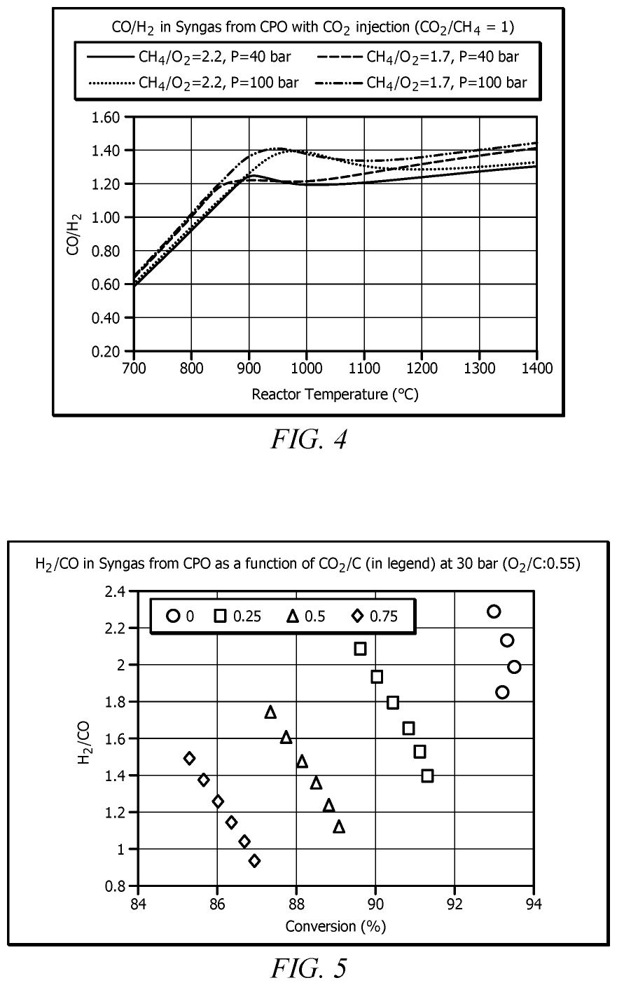

Example 1. A syngas process was simulated as an equilibrium reactor in ASPEN. FIG. 2 is a plot of the molar ratio of carbon monoxide to hydrogen (CO/H₂) in syngas from CPO as a function of reactor temperature without CO₂ injection in the reactant feed for CH₄/O₂ molar ratios of 2.2 and 1.7, and pressures of 40 and 100 bar, which shows the CO/H₂ molar ratios which can be obtained in CPO subject to thermodynamic constraints at different temperatures of CPO reactor 10. FIG. 3 is a plot of the molar ratio of carbon monoxide to hydrogen (CO/H₂) in syngas from CPO as a function of reactor temperature with CO₂ injection for a reactant feed comprising a molar ratio of carbon dioxide to methane (CO₂/CH₄) of 0.5, CH₄/O₂ molar ratios of 2.2 and 1.7, and pressures of 40 and 100 bar. FIG. 4 is a plot of the molar ratio of carbon monoxide to hydrogen (CO/H₂) in syngas from CPO as a function of reactor temperature with CO₂ injection for a reactant feed comprising a molar ratio of carbon dioxide to methane (CO₂/CH₄) of 1, CH₄/O₂ molar ratios of 2.2 and 1.7, and pressures of 40 and 100 bar.

As seen in FIG. 2, above 900° C. and at a low CH₄/O₂ molar ratio synthesis gas having H₂/CO molar ratios less than 2 can be produced. From FIG. 3 and FIG. 4, it is apparent that injecting CO₂ in the reactant feed expands the operability window of CPO to lower temperatures and higher CH₄/O₂ molar ratios. As noted herein, injection of CO₂ also provides for an enhancement in carbon efficiency, because the carbon in the CO₂ is converted to additional CO. As a result more CO will be produced per MMBTU of reactant feed (e.g., natural gas) according to embodiments of this disclosure. This additional CO can contribute to an increase in chemical product throughput at the same flowrate of reactant feed (e.g., natural gas). A similar effect can be obtained by subjecting all or a portion of the synthesis gas in the CPO reactor effluent 15 to reverse WGS in r-WGS reactor 20 by injection of CO₂ 7B in a separate r-WGS reactor 20 downstream of CPO reactor 10. As seen in FIGS. 2-4, the CPO reactor can produce the hydrogen-lean syngas at high pressures (e.g., greater than or equal to about 25, 30, 35, 40, 45, 50 bar) that are required for downstream chemical (e.g., acetic acid, DME) synthesis, thus reducing or eliminating the need for compression of the CPO reactor effluent prior to the downstream synthesis 30.

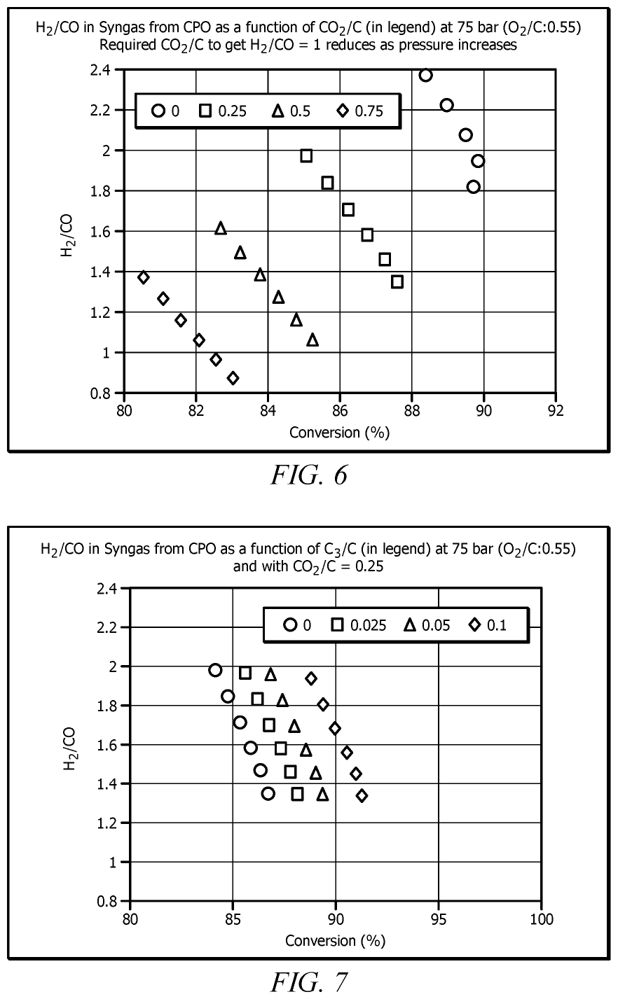

FIG. 5 is a plot showing the molar ratio of carbon monoxide to hydrogen (H₂/CO) in syngas from CPO as a function of the conversion (%) and the molar ratio of carbon dioxide to carbon (CO₂/C) in the reactant feed (in legend) at a pressure of 30 bar and an oxygen to carbon molar ratio (O₂/C) of 0.55. FIG. 6 is a plot showing the molar ratio of carbon monoxide to hydrogen (H₂/CO) in syngas from CPO as a function of the conversion (%) and the molar ratio of carbon dioxide to carbon (CO₂/C) in the reactant feed (in legend) at a pressure of 75 bar and an oxygen to carbon molar ratio (O₂/C) of 0.55. As can be seen from FIGS. 5 and 6, the molar ratio of CO₂/C needed to provide a hydrogen-lean CPO syngas having a H₂/CO molar ratio of 1 is reduced as the pressure increases.

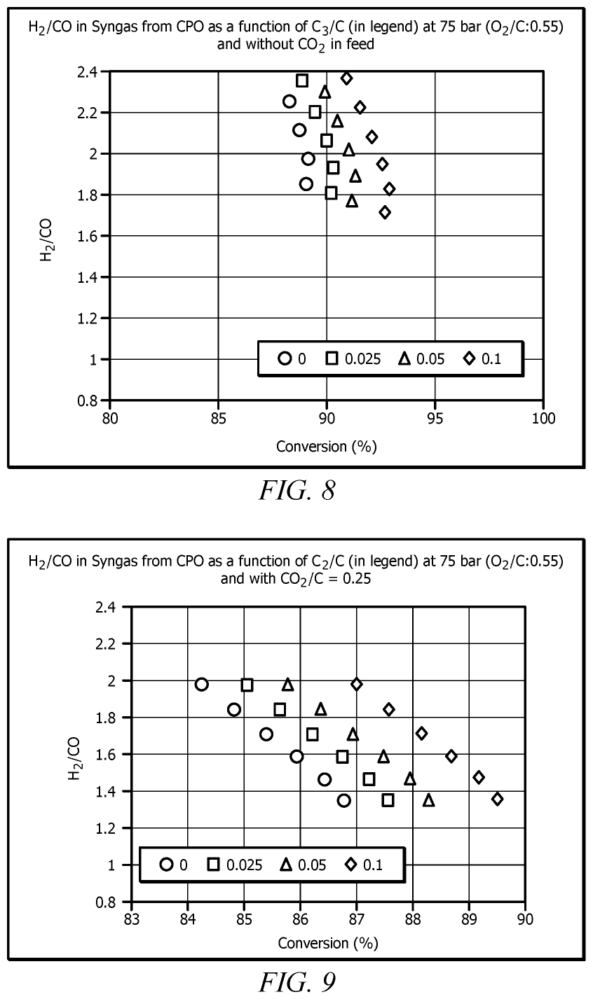

FIG. 7 is a plot showing the molar ratio of carbon monoxide to hydrogen (H₂/CO) in syngas from CPO as a function of the conversion (%) and the molar ratio of hydrocarbons having three carbons (C₃) to carbon (C₃/C) in the reactant feed (in legend) at a pressure of 75 bar, an oxygen to carbon molar ratio (O₂/C) of 0.55, and a carbon dioxide to carbon (CO₂/C) molar ratio of 0.25. FIG. 8 is a plot showing the molar ratio of carbon monoxide to hydrogen (H₂/CO) in syngas from CPO as a function of the conversion (%) and the molar ratio of hydrocarbons having three carbons (C₃) to carbon (C₃/C) in the reactant feed (in legend) at a pressure of 75 bar, an oxygen to carbon molar ratio (O₂/C) of 0.55, and without CO₂ in the reactant feed.

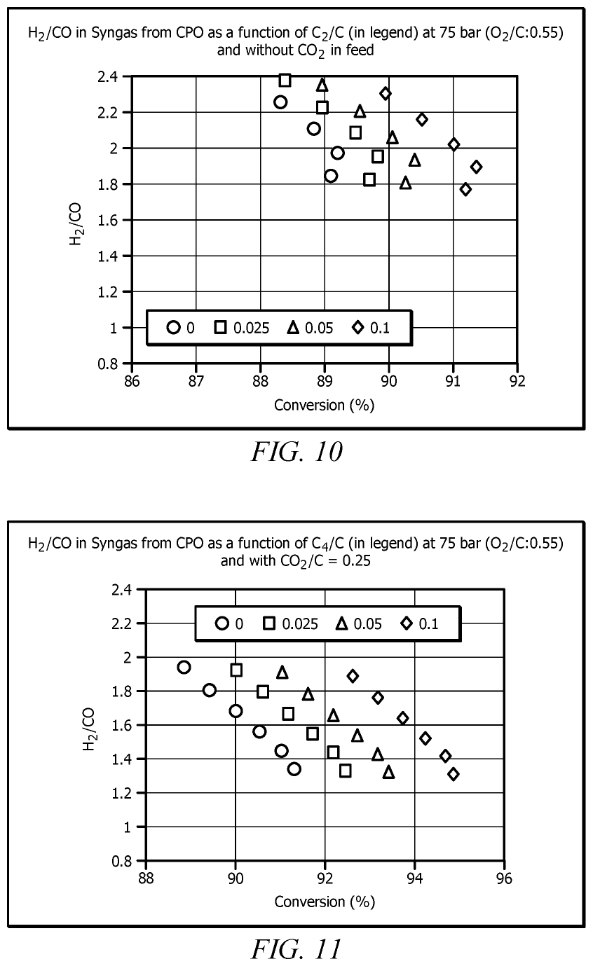

FIG. 9 is a plot showing the molar ratio of carbon monoxide to hydrogen (H₂/CO) in syngas from CPO as a function of the conversion (%) and the molar ratio of hydrocarbons having two carbons (C₂) to carbon (C₂/C) in the reactant feed (in legend) at a pressure of 75 bar, an oxygen to carbon molar ratio (O₂/C) of 0.55, and a CO₂/C molar ratio of 0.25. FIG. 10 is a plot the molar ratio of carbon monoxide to hydrogen (H₂/CO) in syngas from CPO as a function of the conversion (%) and the molar ratio of hydrocarbons having two carbons (C₂) to carbon (C₂/C) in the reactant feed (in legend) at a pressure of 75 bar, an oxygen to carbon molar ratio (O₂/C) of 0.55, and without CO₂ in the reactant feed.

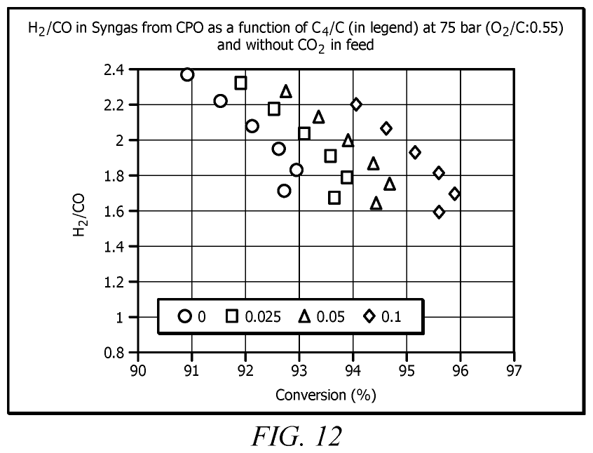

FIG. 11 is a plot showing the molar ratio of carbon monoxide to hydrogen (H₂/CO) in syngas from CPO as a function of the conversion (%) and the molar ratio of hydrocarbons having four carbons (C₄) to carbon (C₄/C) in the reactant feed (in legend) at a pressure of 75 bar, an oxygen to carbon molar ratio (O₂/C) of 0.55, and a carbon dioxide to carbon (CO₂/C) molar ratio of 0.25. FIG. 12 is a plot showing the molar ratio of carbon monoxide to hydrogen (H₂/CO) in syngas from CPO as a function of the conversion (%) and the molar ratio of hydrocarbons having four carbons (C₄) to carbon (C₄/C) in the reactant feed (in legend) at a pressure of 75 bar, an oxygen to carbon molar ratio (O₂/C) of 0.55, and without CO₂ in the reactant feed.

As seen in FIGS. 7 through 12, using reactant feeds 5 comprising higher hydrocarbons (e.g., C₂, C₃, and/or C₄) allows a reduction in the amount of CO₂ utilized to reach a molar ratio of hydrogen to carbon monoxide (H₂/CO) of about 1, and enables production of hydrogen-lean syngas having an H₂/CO molar ratio of about 1 at a higher hydrocarbon conversion to syngas.

While various embodiments have been shown and described, modifications thereof can be made by one skilled in the art without departing from the spirit and teachings of the disclosure. The embodiments described herein are exemplary only, and are not intended to be limiting. Many variations and modifications of the subject matter disclosed herein are possible and are within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R_{L} and an upper limit, R_{U} is disclosed, any number falling within the range is specifically disclosed. In particular, the following numbers within the range are specifically disclosed: R=R_{L}+k*(R_{U}−R_{L}), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present disclosure. Thus, the claims are a further description and are an addition to the embodiments of the present disclosure. The discussion of a reference is not an admission that it is prior art to the present disclosure, especially any reference that may have a publication date after the priority date of this application. The disclosures of all patents, patent applications, and publications cited herein are hereby incorporated by reference, to the extent that they provide exemplary, procedural, or other details supplementary to those set forth herein.

### ADDITIONAL DESCRIPTION

The particular embodiments disclosed above are illustrative only, as the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present disclosure. Alternative embodiments that result from combining, integrating, and/or omitting features of the embodiment(s) are also within the scope of the disclosure. While compositions and methods are described in broader terms of “having”, “comprising,” “containing,” or “including” various components or steps, the compositions and methods can also “consist essentially of” or “consist of” the various components and steps. Use of the term “optionally” with respect to any element of a claim means that the element is required, or alternatively, the element is not required, both alternatives being within the scope of the claim.

Numbers and ranges disclosed above may vary by some amount. Whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. If there is any conflict in the usages of a word or term in this specification and one or more patent or other documents, the definitions that are consistent with this specification should be adopted.

Embodiments disclosed herein include:

A: A process for producing hydrogen-lean syngas comprising reacting, via a catalytic partial oxidation (CPO) reaction, a CPO reactant mixture in a CPO reactor to produce the hydrogen-lean syngas; wherein the CPO reactant mixture comprises hydrocarbons and oxygen; wherein the hydrocarbons comprise greater than or equal to about 3 mol % C₂₊ alkanes; wherein the CPO reactor comprises a CPO catalyst; wherein the hydrogen-lean syngas comprises hydrogen, carbon monoxide, carbon dioxide, water, and unreacted hydrocarbons; and wherein the hydrogen-lean syngas is characterized by a hydrogen to carbon monoxide (H₂/CO) molar ratio of from about 0.8 to about 1.6.

B: A process comprising: (a) reacting, via a catalytic partial oxidation (CPO) reaction, a CPO reactant mixture in a CPO reactor to produce a hydrogen-lean syngas; wherein the CPO reactant mixture comprises hydrocarbons and oxygen; wherein the hydrocarbons comprise greater than or equal to about 3 mol % C₂₊ alkanes; wherein the CPO reactor comprises a CPO catalyst; wherein the hydrogen-lean syngas comprises hydrogen, carbon monoxide, carbon dioxide (CO₂), water, and unreacted hydrocarbons; and wherein the hydrogen-lean syngas is characterized by a hydrogen to carbon monoxide (H₂/CO) molar ratio of from about 0.8 to about 1.6; (b) optionally introducing CO₂ to the CPO reactor, wherein the CPO reactant mixture is characterized by a CO₂ to carbon (CO₂/C) molar ratio in the CPO reactant mixture of greater than or equal to about 0.5:1, wherein the CO₂/C molar ratio refers to the total moles of CO₂ in the reactant mixture divided by the total moles of C in the hydrocarbons in the reactant mixture; and (c) using at least a portion of the hydrogen-lean syngas in a downstream synthesis process, wherein the downstream synthesis process is selected from the group consisting of acetic acid synthesis process; dimethyl ether synthesis process; oxo-synthesis of aliphatic aldehydes and/or alcohols; and combinations thereof.

C: A chemical synthesis system comprising: (a) a catalytic partial oxidation (CPO) reactor comprising a CPO catalyst, and operable to produce a hydrogen-lean syngas from a CPO reactant mixture; wherein the CPO reactant mixture comprises hydrocarbons and oxygen; wherein the hydrocarbons comprise greater than or equal to about 3 mol % C₂₊ alkanes; wherein the hydrogen-lean syngas comprises hydrogen, carbon monoxide, carbon dioxide (CO₂), water, and unreacted hydrocarbons; and wherein the hydrogen-lean syngas is characterized by a hydrogen to carbon monoxide (H₂/CO) molar ratio of from about 0.8 to about 1.6; and (b) a downstream synthesis apparatus configured to produce a chemical product from at least a portion of the hydrogen-lean syngas, wherein the downstream synthesis process is selected from the group consisting of acetic acid synthesis process; dimethyl ether synthesis process; oxo-synthesis of aliphatic aldehydes and/or alcohols; and combinations thereof.

Each of embodiments A, B, and C may have one or more of the following additional elements: Element 1: wherein the hydrocarbons comprise methane, natural gas, natural gas liquids, liquefied petroleum gas (LPG), associated gas, well head gas, enriched gas, paraffins, shale gas, shale liquids, fluid catalytic cracking (FCC) off gas, refinery process gases, refinery off gases, stack gases, fuel gas from a fuel gas header, or combinations thereof. Element 2: wherein the hydrocarbons comprise ethane in an amount of greater than or equal to about 4 mol %. Element 3: wherein the hydrocarbons comprise propane in an amount of greater than or equal to about 4 mol %. Element 4: wherein the hydrocarbons comprise butanes in an amount of greater than or equal to about 3 mol %. Element 5: wherein the hydrocarbons conversion in the CPO reactor is greater than the hydrocarbons conversion in a CPO reactor in an otherwise similar process that produces a hydrogen-lean syngas from hydrocarbons comprising less than about 3 mol % C₂₊ alkanes. Element 6: wherein the CPO reactant mixture further comprises carbon dioxide (CO₂); and wherein the CPO reactant mixture is characterized by a CO₂ to carbon (CO₂/C) molar ratio in the CPO reactant mixture of greater than or equal to about 0.5:1, wherein the CO₂/C molar ratio refers to the total moles of CO₂ in the reactant mixture divided by the total moles of carbon (C) in the hydrocarbons in the reactant mixture. Element 7: wherein the amount of CO₂ in the CPO reactant mixture is lower than the amount of CO₂ in a CPO reactant mixture in an otherwise similar process that produces a hydrogen-lean syngas from hydrocarbons comprising less than about 3 mol % C₂₊ alkanes. Element 8: wherein the CPO reactor is characterized by at least one CPO operational parameter selected from the group consisting of a CPO reactant temperature of from about 100° C. to about 500° C.; a CPO pressure of from about 20 barg to about 80 barg; a CPO contact time of from about 0.001 milliseconds (ms) to about 5 seconds (s); a carbon to oxygen (C/O) molar ratio in the CPO reactant mixture of from about 0.5:1 to about 3:1, wherein the C/O molar ratio refers to the total moles of carbon (C) in the hydrocarbons in the reactant mixture divided by the total moles of oxygen (O₂) in the reactant mixture; a steam to carbon (S/C) molar ratio in the CPO reactant mixture of less than about 0.6:1, wherein the S/C molar ratio refers to the total moles of water (H₂O) in the reactant mixture divided by the total moles of carbon (C) in the hydrocarbons in the reactant mixture; and combinations thereof. Element 9: further comprising: (i) recovering a CPO reactor effluent from the CPO reactor, wherein the CPO reactor effluent comprises hydrogen, carbon monoxide, carbon dioxide, water, and unreacted hydrocarbons, and wherein the CPO reactor effluent is characterized by a H₂/CO molar ratio of greater than about 1.6; and (ii) feeding at least a portion of the CPO reactor effluent to a reverse water-gas shift (r-WGS) reactor to produce the hydrogen-lean syngas, wherein a portion of the hydrogen of the CPO reactor effluent reacts with carbon dioxide via a r-WGS reaction to produce water and carbon monoxide. Element 10: further comprising introducing additional carbon dioxide to the r-WGS reactor. Element 11: further comprising: (a) recovering a r-WGS reactor effluent from the r-WGS reactor, wherein the r-WGS reactor effluent comprises hydrogen, carbon monoxide, carbon dioxide, water, and unreacted hydrocarbons; and (b) removing at least a portion of the water from the r-WGS reactor effluent to yield the hydrogen-lean syngas, wherein the amount of water in the r-WGS reactor effluent is greater than the amount of water in the hydrogen-lean syngas. Element 12: further comprising: (1) contacting a portion of the CPO reactor effluent with at least a portion of the r-WGS reactor effluent to produce a combined effluent stream; and (2) removing at least a portion of the water from the combined effluent stream to yield the hydrogen-lean syngas, wherein the amount of water in the combined effluent stream is greater than the amount of water in the hydrogen-lean syngas. Element 13: excluding a step of introducing at least a portion of the CPO reactor effluent and/or at least a portion of the hydrogen-lean syngas to a hydrogen recovery unit to decrease the amount of hydrogen in the CPO reactor effluent and/or the hydrogen-lean syngas, respectively. Element 14: wherein a portion of the carbon dioxide in the CPO reactor undergoes a reverse water-gas shift (r-WGS) reaction, thereby decreasing the amount of hydrogen in the hydrogen-lean syngas. Element 15: further comprising using at least a portion of the hydrogen-lean syngas in a downstream synthesis process. Element 16: wherein the downstream synthesis process is selected from the group consisting of acetic acid synthesis process; dimethyl ether synthesis process; oxo-synthesis of aliphatic aldehydes and/or alcohols; and combinations thereof. Element 17: wherein (i) the hydrocarbons conversion in the CPO reactor is greater than the hydrocarbons conversion in a CPO reactor in an otherwise similar process that produces a hydrogen-lean syngas from hydrocarbons comprising less than about 3 mol % C₂₊ alkanes; and/or (ii) the amount of CO₂ in the CPO reactant mixture is lower than the amount of CO₂ in a CPO reactant mixture in an otherwise similar process that produces a hydrogen-lean syngas from hydrocarbons comprising less than about 3 mol % C₂₊ alkanes. Element 18: wherein the CPO reactor is characterized by at least one CPO operational parameter selected from the group consisting of a CPO reactant mixture temperature of from about 100° C. to about 500° C.; a CPO pressure of from about 25 barg to about 80 barg; a CPO contact time of from about 0.001 milliseconds (ms) to about 5 seconds (s); a carbon to oxygen (C/O) molar ratio in the CPO reactant mixture of from about 0.5:1 to about 2:1, wherein the C/O molar ratio refers to the total moles of carbon (C) in the hydrocarbons in the reactant mixture divided by the total moles of oxygen (O₂) in the reactant mixture; a steam to carbon (S/C) molar ratio in the CPO reactant mixture of less than about 0.25:1, wherein the S/C molar ratio refers to the total moles of water (H₂O) in the reactant mixture divided by the total moles of carbon (C) in the hydrocarbons in the reactant mixture; and combinations thereof. Element 19: (i) comprising no apparatus for altering the H₂/CO molar ratio of the hydrogen-lean syngas between the CPO reactor and the downstream synthesis apparatus; (ii) comprising a reverse water gas shift apparatus as a sole apparatus for altering the H₂/CO molar ratio of the hydrogen-lean syngas prior to the downstream synthesis apparatus; or (iii) comprising no apparatus configured to remove a hydrogen stream from the hydrogen-lean syngas between the CPO reactor and the downstream synthesis apparatus.

While preferred embodiments of the invention have been shown and described, modifications thereof can be made by one skilled in the art without departing from the teachings of this disclosure. The embodiments described herein are exemplary only, and are not intended to be limiting. Many variations and modifications of the invention disclosed herein are possible and are within the scope of the invention.

Numerous other modifications, equivalents, and alternatives, will become apparent to those skilled in the art once the above disclosure is fully appreciated. It is intended that the following claims be interpreted to embrace all such modifications, equivalents, and alternatives where applicable. Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

1. A process for producing hydrogen-lean syngas comprising reacting, via a catalytic partial oxidation (CPO) reaction, a CPO reactant mixture in a CPO reactor to produce the hydrogen-lean syngas; wherein the CPO reactant mixture comprises hydrocarbons and oxygen; wherein the hydrocarbons comprise greater than or equal to about 3 mol % C2+ alkanes; wherein the CPO reactor comprises a CPO catalyst; wherein the hydrogen-lean syngas comprises hydrogen, carbon monoxide, carbon dioxide, water, and unreacted hydrocarbons; and wherein the hydrogen-lean syngas is characterized by a hydrogen to carbon monoxide (H2/CO) molar ratio of from about 0.8 to about 1.6.

2. The process of claim 1, wherein the hydrocarbons comprise methane, natural gas, natural gas liquids, liquefied petroleum gas (LPG), associated gas, well head gas, enriched gas, paraffins, shale gas, shale liquids, fluid catalytic cracking (FCC) off gas, refinery process gases, refinery off gases, stack gases, fuel gas from a fuel gas header, or combinations thereof.

3. The process of claim 1, wherein the hydrocarbons comprise ethane in an amount of greater than or equal to about 4 mol %; wherein the hydrocarbons comprise propane in an amount of greater than or equal to about 4 mol %; wherein the hydrocarbons comprise butanes in an amount of greater than or equal to about 3 mol %; or a combination thereof.

4. The process of claim 1, wherein the hydrocarbons conversion in the CPO reactor is greater than the hydrocarbons conversion in a CPO reactor in an otherwise similar process that produces a hydrogen-lean syngas from hydrocarbons comprising less than about 3 mol % C2+ alkanes.

5. The process of claim 1, wherein the CPO reactant mixture further comprises carbon dioxide (C02); and wherein the CPO reactant mixture is characterized by a C02 to carbon (C02/C) molar ratio in the CPO reactant mixture of greater than or equal to about 0.5:1, wherein the CO 2/C molar ratio refers to the total moles of C02 in the reactant mixture divided by the total moles of carbon (C) in the hydrocarbons in the reactant mixture.

6. The process of claim 5, wherein the amount of C02 in the CPO reactant mixture is lower than the amount of C02 in a CPO reactant mixture in an otherwise similar process that produces a hydrogen-lean syngas from hydrocarbons comprising less than about 3 mol % C2+ alkanes.

7. The process of claim 1, wherein the CPO reactor is characterized by at least one CPO operational parameter selected from the group consisting of a CPO reactant temperature of from about 100° C. to about 500° C.; a CPO pressure of from about 20 barg to about 80 barg; a CPO contact time of from about 0.001 milliseconds (ms) to about 5 seconds (s); a carbon to oxygen (C/O) molar ratio in the CPO reactant mixture of from about 0.5:1 to about 3:1, wherein the C/O molar ratio refers to the total moles of carbon (C) in the hydrocarbons in the reactant mixture divided by the total moles of oxygen (02) in the reactant mixture; a steam to carbon (S/C) molar ratio in the CPO reactant mixture of less than about 0.6:1, wherein the S/C molar ratio refers to the total moles of water (H20) in the reactant mixture divided by the total moles of carbon (C) in the hydrocarbons in the reactant mixture; and combinations thereof.

8. The process of claim 1 further comprising: (i) recovering a CPO reactor effluent from the CPO reactor, wherein the CPO reactor effluent comprises hydrogen, carbon monoxide, carbon dioxide, water, and unreacted hydrocarbons, and wherein the CPO reactor effluent is characterized by a H2/CO molar ratio of greater than about 1.6; and (ii) feeding at least a portion of the CPO reactor effluent to a reverse water-gas shift (r-WGS) reactor to produce the hydrogen-lean syngas, wherein a portion of the hydrogen of the CPO reactor effluent reacts with carbon dioxide via a r-WGS reaction to produce water and carbon monoxide.

9. The process of claim 8 further comprising introducing additional carbon dioxide to the r-WGS reactor.

10. The process of claim 9 further comprising: (a) recovering a r-WGS reactor effluent from the r-WGS reactor, wherein the r-WGS reactor effluent comprises hydrogen, carbon monoxide, carbon dioxide, water, and unreacted hydrocarbons; and (b) removing at least a portion of the water from the r-WGS reactor effluent to yield the hydrogen-lean syngas, wherein the amount of water in the r-WGS reactor effluent is greater than the amount of water in the hydrogen-lean syngas.

11. The process of claim 10 further comprising: (1) contacting a portion of the CPO reactor effluent with at least a portion of the r-WGS reactor effluent to produce a combined effluent stream; and (2) removing at least a portion of the water from the combined effluent stream to yield the hydrogen-lean syngas, wherein the amount of water in the combined effluent stream is greater than the amount of water in the hydrogen-lean syngas.

12. The process of claim 11 excluding a step of introducing at least a portion of the CPO reactor effluent and/or at least a portion of the hydrogen-lean syngas to a hydrogen recovery unit to decrease the amount of hydrogen in the CPO reactor effluent and/or the hydrogen-lean syngas, respectively.

13. The process of claim 1, wherein a portion of the carbon dioxide in the CPO reactor undergoes a reverse water-gas shift (r-WGS) reaction, thereby decreasing the amount of hydrogen in the hydrogen-lean syngas.

14. The process of claim 1 further comprising using at least a portion of the hydrogen-lean syngas in a downstream synthesis process.

15. The process of claim 14, wherein the downstream synthesis process is selected from the group consisting of acetic acid synthesis process; dimethyl ether synthesis process; oxo-synthesis of aliphatic aldehydes and/or alcohols; and combinations thereof.

16. A process comprising:
(a) reacting, via a catalytic partial oxidation (CPO) reaction, a CPO reactant mixture in a CPO reactor to produce a hydrogen-lean syngas; wherein the CPO reactant mixture comprises hydrocarbons and oxygen; wherein the hydrocarbons comprise greater than or equal to about 3 mol % C2+ alkanes; wherein the CPO reactor comprises a CPO catalyst; wherein the hydrogen-lean syngas comprises hydrogen, carbon monoxide, carbon dioxide (C02), water, and unreacted hydrocarbons; and wherein the hydrogen-lean syngas is characterized by a hydrogen to carbon monoxide (H2/CO) molar ratio of from about 0.8 to about 1.6;
(b) optionally introducing C02 to the CPO reactor, wherein the CPO reactant mixture is characterized by a C02 to carbon (C02/C) molar ratio in the CPO reactant mixture of greater than or equal to about 0.5:1, wherein the CO 2/C molar ratio refers to the total moles of C02 in the reactant mixture divided by the total moles of carbon (C) in the hydrocarbons in the reactant mixture; and
(c) using at least a portion of the hydrogen-lean syngas in a downstream synthesis process, wherein the downstream synthesis process is selected from the group consisting of acetic acid synthesis process; dimethyl ether synthesis process; oxo-synthesis of aliphatic aldehydes and/or alcohols; and combinations thereof.

17. The process of claim 16, wherein (i) the hydrocarbons conversion in the CPO reactor is greater than the hydrocarbons conversion in a CPO reactor in an otherwise similar process that produces a hydrogen-lean syngas from hydrocarbons comprising less than about 3 mol % C2+ alkanes; and/or (ii) the amount of C02 in the CPO reactant mixture is lower than the amount of C02 in a CPO reactant mixture in an otherwise similar process that produces a hydrogen-lean syngas from hydrocarbons comprising less than about 3 mol % C2+ alkanes.

18. The process of claim 16, wherein the CPO reactor is characterized by at least one CPO operational parameter selected from the group consisting of a CPO reactant mixture temperature of from about 100° C. to about 500° C.; a CPO pressure of from about 25 barg to about 80 barg; a CPO contact time of from about 0.001 milliseconds (ms) to about 5 seconds (s); a carbon to oxygen (C/O) molar ratio in the CPO reactant mixture of from about 0.5:1 to about 2:1, wherein the C/O molar ratio refers to the total moles of carbon (C) in the hydrocarbons in the reactant mixture divided by the total moles of oxygen (02) in the reactant mixture; a steam to carbon (S/C) molar ratio in the CPO reactant mixture of less than about 0.25:1, wherein the S/C molar ratio refers to the total moles of water (H20) in the reactant mixture divided by the total moles of carbon (C) in the hydrocarbons in the reactant mixture; and combinations thereof.

19. A chemical synthesis system comprising:
(a) a catalytic partial oxidation (CPO) reactor comprising a CPO catalyst, and operable to produce a hydrogen-lean syngas from a CPO reactant mixture; wherein the CPO reactant mixture comprises hydrocarbons and oxygen; wherein the hydrocarbons comprise greater than or equal to about 3 mol % C2+ alkanes; wherein the hydrogen-lean syngas comprises hydrogen, carbon monoxide, carbon dioxide (C02), water, and unreacted hydrocarbons; and wherein the hydrogen-lean syngas is characterized by a hydrogen to carbon monoxide (H2/CO) molar ratio of from about 0.8 to about 1.6; and
(b) a downstream synthesis apparatus configured to produce a chemical product from at least a portion of the hydrogen-lean syngas, wherein the downstream synthesis process is selected from the group consisting of acetic acid synthesis process; dimethyl ether synthesis process; oxo-synthesis of aliphatic aldehydes and/or alcohols; and combinations thereof.

20. The chemical synthesis system of claim 19 further comprising:
(i) no apparatus for altering the H2/CO molar ratio of the hydrogen-lean syngas between the CPO reactor and the downstream synthesis apparatus;
(ii) a reverse water gas shift apparatus as a sole apparatus for altering the H2/CO molar ratio of the hydrogen-lean syngas prior to the downstream synthesis apparatus; or
(iii) no apparatus configured to remove a hydrogen stream from the hydrogen-lean syngas between the CPO reactor and the downstream synthesis apparatus.
